(19) United States
(12) Patent Application Publication
(10) Pub. No.: US 2016/0228384 A1
(43) Pub. Date: Aug. 11, 2016

(54) EMULSION CONTAINING TWO OILS AND STABILIZERS

(71) Applicant: NANOMEDEX, LLC, Madison, WI (US)

(72) Inventor: David Lynn Cooper, Homosassa, FL (US)

(21) Appl. No.: 15/015,541

(22) Filed: Feb. 4, 2016

Related U.S. Application Data
(60) Provisional application No. 62/112,426, filed on Feb. 5, 2015.

(51) Int. Cl. A61K 31/05; A61K 47/44; A61K 47/14; A61K 9/107

(52) U.S. Cl. CPC A61K 31/05; A61K 9/1075; A61K 47/44; A61K 47/14

(57) ABSTRACT

The present invention relates to improved formulations for administration lipophilic drugs, and in particular to improved propofol formulations. Emulsion of the present invention preferably comprise oil droplets of a mean oil particle diameter of 80-300 nanometers; and a continuous aqueous phase comprising a lipophilic drug in an amount 0.5-5.0% by weight relative to the weight of the total emulsion, wherein said lipophilic drug has a solubility in water of less than 1 mg/mL; a primary oil physiologically suitable for parenteral administration to a mammal comprising plant-derived biocompatible long chain triglycerides; and a secondary oil comprising an ethyl ester of a saturated, unbranched carboxylic acid of 4-8 carbon atoms or an unbranched alkyl esters of acetic acid, said alkyl residue having 4-8 carbon atoms, or combination thereof, the combined percentage by weight of the oil components not exceeding about 10 percent.

### CROSS-REFERENCE TO RELATED APPLICATIONS

The present application claims the priority benefit of U.S. Provisional Patent Application 62/112,426, filed Feb. 5, 2015, which is incorporated by reference in its entirety.

### FIELD OF THE INVENTION

The present invention relates to improved formulations for administration lipophilic drugs, and in particular to improved propofol formulations.

### BACKGROUND OF THE INVENTION

Many widely used drugs have solubility in water of less than 1 mg/mL. As a result, effective doses of these poorly water-soluble, lipophilic drugs must be delivered in formulations that are oil-in-water emulsions. Propofol is an example of a member of this class of drugs. Other examples of poorly water-soluble, lipophilic drugs include: cancer chemotherapeutic agents (oncology drugs) such as doxorubicin, camptothecin, paclitaxel, etoposide, and daunorubicin; antibiotics such as the macrolides (e.g., erythromycin, solithromycin, azithromycin, and so forth); antifungal therapies such as the amphotericin B's, voriconazole, and posaconazole; immunotherapeutic agents such as cyclosporine, muramyl peptides, rapamycin, levamisole, and vaccines; and immuno-adjuvants such as squalene.

Propofol (2,6-diisopropylphenol) is a short-acting intravenous anesthetic agent used for the induction of general anesthesia in adult patients and pediatric patients older than 3 years of age; maintenance of general anesthesia in adult patients and pediatric patients older than 2 months of age; and intensive care unit (ICU) sedation for intubated, mechanically ventilated adults. Propofol is approved for the induction and maintenance of anesthesia in more than 50 countries around the world, including the United States.

Propofol has very low solubility in water as evidenced by an octanol/water partition coefficient of 6761:1 at a solution pH of 6 to 8.5. Therefore, the drug is conventionally provided as an aqueous emulsion containing 1% propofol solubilized in a long-chain triglyceride (LCT; e.g., 10% soybean oil), purified egg phospholipids, and glycerol. Alternatively, propofol is provided as an aqueous emulsion of 1% propofol solubilized in 5% medium-chain triglycerides/5% long-chain triglycerides, egg phospholipids, and 2.25% glycerol. Conventional emulsions have a pH between 6.0 and 8.5. One of the conventional emulsions, Diprivan® (Fresenius Kabi, APP), contains EDTA as an antimicrobial agent. Other conventional formulations contain antimicrobial agents such as benzyl alcohol.

Propofol emulsions for injection usually are made by diluting propofol in the oil components of the formulation, followed by mixing with aqueous solutions of the other formulation components and emulsification into oil-in-water emulsions. The oil components are typically long-chain triglycerides (LCT) or medium-chain triglycerides (MCT). A LCT oil is a vegetable oil composed of glycerol esters of fatty acids having a chain length of 14 to 22 carbons. The fatty acids in a LCT include both saturated fatty acids and unsaturated fatty acids. Because the vegetable oil is obtained from plants, the fatty acid composition of an LCT varies. A MCT oil is a synthetic oil which is obtained by hydrolysis of the fixed oil extracted from the hard, dried fraction of the endosperm of Cocos nucifera L., followed by re-esterification of the purified fatty acids with glycerol to produce MCT oils that are mainly glycerol esters of fatty acids having a chain length of six to twelve carbons including hexanoic or caproic acid (6 carbons), octanoate or caprylic acid (eight carbons), decanoic or capric acid (10 carbons), and dodecanoic or lauric acid (12 carbons) in a ratio of approximately 2:55:42:1. (source: http://www.pdrhealth.com) In general, a solution of propofol in oil is added to an aqueous solution of the other formulation components and mixed to form a coarse emulsion. The coarse emulsion is then emulsified to form an oil-in-water macroemulsion, an oil-in-water nanoemulsion, or an oil-in-water microemulsion. Some general properties of these types of emulsions are summarized in Table 1, below.

TABLE 1
Selected properties of oil-in-water emulsions

| Type of Emulsion | Average Particle Size* | Appearance |
|---|---|---|
| Macroemulsion | >400 nm (0.4 microns) | White |
| Nanoemulsion | 100-400 nm | Blue white |
| Microemulsion | <100 nm | Translucent |
| | <50 nm | Transparent |

*Mean oil particle diameter

Aside from the hypotension and transient apnea following induction doses, one of its most frequent side-effects of intravenous administration of conventional propofol emulsions is pain on injection, especially when the injection site is located in smaller veins. Although injection site pain can be mitigated by pretreatment or mixing with intravenous lidocaine, for many years pharmaceutical formulators have attempted to reduce or eliminate the pain produced by propofol through reformulation. [See, for example, M. T. Baker et al., “Propofol: The challenges of reformulation,” Anesthesiology 105(4): 860-876, 2005.] Since the pain produced by propofol may be associated with the quantity of drug in the aqueous phase of the emulsion, a widely utilized criterion that is used to evaluate the suitability of new formulations for intravenous administration is to achieve a lower concentration of propofol in the aqueous phase of the emulsion. [See, for example, L Jabota, et al., Prevention of pain on injection of propofol: systematic review and meta-analysis,” BMJ|Online First|bmj.com, BMJ 2011:342:d1110, doi:10:1136/bmj.d1110.] For example, conventional LCT-propofol emulsions such as Diprivan® (Fresenius Kabi USA) have a concentration of propofol in the aqueous phase of the emulsion of 18.6±0.6 μg/mL, whereas conventional LCT/MCT-propofol emulsions have a concentration of propofol equal to about 14±0.5 μg/mL in the aqueous phase of the emulsion. Reduction in the irritation of the vascular endothelium at the injection site has been attributed to a reduced concentration of propofol in the aqueous phase of the emulsion. [Müller, RH; Harnisch, S. Physico-chemical characterization of propofol-loaded emulsions and interaction with plasma proteins. Eur J Hosp Pharm 2000; 6: 24-31.]

There are many strategies proposed in the patent literature for minimizing adverse local effects including pain and thrombosis associated with propofol administration. Some approaches seek to ameliorate localized trauma by affecting the local physiology; and others by masking the drug to protect contact tissues. In Zhang (U.S. Patent Application 2014/0031435) and Emcure (WO 2014/033751) a buffering system is disclosed which maintains pH about 6.5. A propofol formulation containing a block copolymer, polyethylene glycol, and propylene glycol is described in EP 1 539 122, intended to attenuate the immediate effects of the drug until it reaches the blood stream. Roewer, et al. (U.S. Patent Application 2013/0316976) discloses use of cyclodextrin as a binding agent to protect contact tissues. U.S. Pat. No. 7,138,387 (Pai, et al.) describes a variant utilizing hydroxypropyl-beta-cyclodextrin. U.S. Pat. No. 7,915,317 discloses a propofol formulation containing polyethylene glycol and a poloxamer, again in an attempt to entrain the drug in a polymer matrix.

### SUMMARY OF THE INVENTION

The present invention provides a sterile, stable, pharmaceutical oil-in-water emulsion composition of a lipophilic drug having a solubility in water of less than 1 mg/mL. An emulsion of the invention is suitable for intravenous administration and has a low concentration of the lipophilic drug in the aqueous phase of the emulsion that overcomes the drawbacks of prior art compositions.

The present invention provides a stable, intravenously administrable, lipophilic drug-containing, oil-in-water emulsion composition, comprising a primary oil and a secondary oil, wherein the primary oil is selected from a long-chain or medium-chain triglyceride oil; the secondary oil is a monoester of a medium-chain fatty acids; emulsifiers selected from natural or synthetic emulsification agents; tonicity modifying agents; and water.

The present invention is a stable lipophilic drug-containing emulsion having oil droplets of a mean oil particle diameter of 80-300 nanometers and a continuous aqueous phase containing the following components: lipophilic drug in an amount 0.1-5.0% by weight relative to the weight of the total emulsion, a primary oil physiologically suitable and biocompatible with parenteral administration to mammals, and consisting of a plant-derived long chain triglyceride oil such as vegetable oils, almond oil, apricot kernel oil, avocado oil, canola oil, hazelnut oil, coconut oil, mustard oil, oat oil, peanut oil, rice bran oil, safflower oil olive oil, soybean oil, and sunflower oil. A secondary oil of lower molecular weight in which the lipophilic drug is more soluble is also provided and comprises an ethyl ester of a saturated, unbranched carboxylic acid of 4-8 carbon atoms or an unbranched alkyl ester of acetic acid having 4-8 carbon atoms, or a combination thereof, the secondary oil content not exceeding about 50 percent by weight of the total oil components. For example, the secondary oil may be butyl acetate, hexyl acetate, octyl acetate, ethyl butyrate, ethyl hexanoate, or ethyl octanoate.

The preferred formulation also includes stabilizer surfactants of two types: ionic and non-ionic. The ionic surfactants may be sodium myristate, sodium palmitate, sodium palmitoleate, sodium stearate, sodium oleate, sodium linoleate, sodium arachidate, and sodium behenate. The non-ionic surfactants may be a polaxamer having hydroxyl, carboxylate, sulfate, ester, sugar, or amino end-groups. The ionic surfactant is present in an amount 0.00001-0.001% by weight relative to relative to the total weight of the emulsion, and the non-ionic surfactant is present in an amount 1.0-3.0% by weight relative to the total weight of the emulsion.

In addition, a tonicity agent is included in the formulation, in an amount of 2.0-2.5% by weight relative to total emulsion weight, to prevent osmotic disruption of cells that come into contact with the formulation. Preferred tonicity agents are glycerin, dextrose, and mannitol.

It is important in this invention that the secondary oil has a solubility in water of less than 0.75 weight percent and a water solubility in the secondary oil of less than 1.0 weight percent. It is also essential that solubility of the lipophilic drug is greater in the secondary oil than in the primary oil, so that the secondary oil acts as a differential solubility bridge to the primary oil.

Accordingly, in some preferred embodiments, the present invention provides a stable lipophilic drug-containing emulsion comprising: oil droplets of a mean oil particle diameter of 80-300 nanometers; and a continuous aqueous phase comprising: a lipophilic drug in an amount 0.5-5.0% by weight relative to the weight of the total emulsion, wherein said lipophilic drug has a solubility in water of less than 1 mg/mL; a primary oil physiologically suitable for parenteral administration to a mammal comprising plant-derived biocompatible long chain triglycerides; and a secondary oil comprising an ethyl ester of a saturated, unbranched carboxylic acid of 4-8 carbon atoms or an unbranched alkyl esters of acetic acid, said alkyl residue having 4-8 carbon atoms, or combination thereof, the combined percentage by weight of the oil components not exceeding about 10 percent. In some particularly preferred embodiments, the lipophilic drug is propofol. In some embodiments, the continuous aqueous phase further comprises an ionic surfactant in an amount 0.00001-0.001% by weight relative to the total weight of the emulsion. In some embodiments, the continuous aqueous phase further comprises a non-ionic synthetic surfactant in an amount 1.0-3.0% by weight relative to the total weight of the emulsion. In some embodiments, the continuous aqueous phase further comprises a tonicity agent in an amount of 2.0-2.5% by weight relative to the total weight of the emulsion. In some embodiments, the continuous aqueous phase further comprises water to adjust the concentrations of components to the ranges specified. In some embodiments, the primary oil is selected from the group consisting of vegetable oils, almond oil, apricot kernel oil, avocado oil, canola oil, hazelnut oil, mustard oil, coconut oil, oat oil, olive oil, peanut oil, rice bran oil, safflower oil, sesame oil, soybean oil, and sunflower oil. In some embodiments, the secondary oil is butyl acetate, hexyl acetate, octyl acetate, ethyl butyrate, ethyl hexanoate, and ethyl octanoate. In some embodiments, the secondary oil has a solubility in water of less than 0.75 weight percent, and a solubility of less than 1.0 weight percent water in said secondary oil. In some embodiments, the ionic surfactant is selected from the group consisting of sodium myristate, sodium palmitate, sodium palmitoleate, sodium stearate, sodium oleate, sodium linoleate, sodium arachidate, and sodium behenate. In some embodiments, non-ionic surfactant is a poloxamer having hydroxyl, carboxylate, sulfate, ester, sugar, or amino end-groups. In some embodiments, the tonicity agent is glycerin, dextrose, or mannitol. In some embodiments, the lipophilic drug, preferably propofol, is 99.99 percent partitioned into the oil phase of said emulsion, and is present in the aqueous phase of said total emulsion at a concentration of less than about 15 micrograms per milliliter. In some embodiments, the emulsion is essentially free of one or more agents selected from the group consisting of EDTA, egg lecithin and benzyl alcohol. In some embodiments, the pH of the continuous aqueous phase is from pH 4.0 to 8.0. In some embodiments, the emulsion is stable at room temperature for a period selected from the group consisting of at least 6 months, at 12 months, at least 18 months and at least 24 months up to a total of about 36 months.

The method of preparing the emulsions involves dissolving the lipophilic drug in either the primary or secondary oil, or both; dissolving the surfactants into an aqueous stock solution utilizing heat and agitation, as needed; combining the aqueous stock solutions and the oil phase in predetermined proportions with stirring. A tonicity agent is dissolved in water with water volume adjustments to result in the final desired concentration ranges of the components. The mixture is then subjected to a first sonication to produce a coarse emulsion and then to a second sonication to achieve a mean particle diameter of 100-300 nanometers, more preferably 200-300 nm. The resulting emulsion may then be aliquotted into containers, sealed, and sterilized by conventional means such as autoclaving. This obviates any need for inclusion of an antimicrobial or other anti-pathogenic agent in the formulation.

Accordingly, in some embodiments, the present invention provides methods of preparing a stable lipophilic drug-containing emulsion having a lipophilic phase comprising a primary and a secondary oil, and an aqueous phase comprising: dissolving the lipophilic drug in the primary oil or the secondary oil, or both, and combining into a single oil phase; combining aqueous stock solutions and the oil phase in predetermined proportions to provide a mixture; adding water to adjust the concentrations of the components to the desired ranges in the mixture; and emulsifying the mixture under conditions to obtain an emulsion having a 100 to 300 nanometer mean oil particle diameter. In some embodiments, the lipophilic drug is preferably propofol. In some embodiments, the emulsifying further comprises subjecting the oil and water mixture to a first sonication to obtain a coarse emulsion. In some embodiments, the emulsifying further comprises subjecting the coarse emulsion to a second sonication. In some embodiments, the methods further comprise dissolving surfactants into the aqueous phase stock solutions with heat and agitation. In some embodiments, the methods further comprise dissolving a tonicity agent into the aqueous phase stock solution. In some embodiments, the methods further comprise aliquotting the emulsions into containers and sealing. In some embodiments, the methods further comprise sterilizing the contents of the containers by conventional means without addition of anti-pathogenic agents.

### DESCRIPTION OF THE DRAWINGS

FIG. 1 is a graph comparing the time profiles and extents of cream height growth over time for 10% oil-in-water emulsions containing 1% propofol. The emulsions were prepared using oil phases composed of ethyl butyrate; isopropyl myristate; isopropyl palmitate; octanoic acid; or an oil made up in equal parts of soybean oil and ethyl butyrate (50/50 Mix Soy/EB).

FIG. 2A-E is made up of photographs of experimental emulsions containing 1% propofol. The photographs were taken about 12 days after the time of emulsion preparation (May 5, 2013) and at later dates during 2013. The photographs illustrate the Ostwald ripening, creaming and instability that were exhibited by some of the experimental emulsions.

FIG. 3A-B is made up of photographs of a conventional 10% soybean oil emulsion containing 1% propofol. The photograph on the left was taken on the day of preparation (May 5, 2013) and that on the right was taken after approximately four months of storage at ambient temperatures. The absence of creaming confirms that the conventional emulsion was stable.

FIG. 4A-B is made up of photographs of an emulsion of the invention containing 1% propofol. The 10% oil phase was of the emulsion was made up of equal parts soybean oil and ethyl butyrate (EB). The photograph on the left was taken on the day of preparation (May 5, 2013) and that on the right was taken after approximately four months of storage at ambient temperatures. The absence of creaming confirms that the emulsion was stable.

FIG. 5 is a graph showing the effects of repeated freeze-thaw cycles on the mean oil droplet diameter of emulsions of the invention and Diprivan (a conventional emulsion). Each emulsion contained 1% propofol. The composition of the oil phase of the experimental emulsions of the invention is expressed as a percentage by weight of the entire emulsion composition and was 10% olive oil/0% ethyl butyrate (10 Olive 0 EB); 10% soybean oil/0% ethyl butyrate (10 Soy 0 EB); 5% olive oil/5% ethyl butyrate (5 Olive 5 EB); or 5% soybean oil/5% ethyl butyrate (5 Soy 5 EB).

FIG. 6 is a graph illustrating the effects of repeated freeze-thaw cycles on the mean oil droplet diameter of emulsions of the invention and Diprivan (a conventional emulsion). Each emulsion contained 1% propofol. The composition of the oil phase of the experimental emulsions of the invention is expressed as a percentage by weight of the entire emulsion composition and was 5% soybean oil/5% ethyl butyrate (5 Soy 5 EB) and 1% of one of three non-ionic surfactants (Pluronic F68, Tween 80, or Brij 78).

FIG. 7 is a graph showing the effects of repeated freeze-thaw cycles on the mean oil droplet diameter of emulsions of the invention and Diprivan (a conventional emulsion). Each emulsion contained 1% propofol. The composition of the oil phase of the experimental emulsions of the invention is expressed as a percentage by weight of the entire emulsion composition and was 5% olive oil/5% ethyl butyrate (5 Olive 5 EB) or 5% soybean oil/5% ethyl butyrate (5 Soy 5 EB). In addition, the emulsions of the invention contained either 1% or 3% of the non-ionic surfactant Pluronic F68 (Plu).

FIG. 8 is a graph illustrating the changes in propofol (PF) concentration (in pg/mL) of the aqueous phase of emulsions of the invention as the composition of the oil phase was changed from 100% primary oil (in this example, soybean oil or olive oil) to (100−x) % primary oil and x % of the secondary oil ethyl butyrate (EB) in the primary oil. (The percentage x is defined on the x-axis of the graph.)

FIG. 9 is a photograph of a cross-section of the left cephalic vein of dog 1002, an animal that received a 5 mg/kg dose of Macrofol as described in Example 6. The section was proximal to the catheter tip. This photograph exemplifies the histologic findings of all slides from this study except for those shown in FIGS. 10 and 11. The findings were deemed unremarkable and reported as nonspecific findings (NSF).

FIG. 10 is a photograph of a cross-section of the cephalic vein of dog 2503, an animal that received a 5 mg/kg dose of Diprivan as described in Example 6. This photograph exemplifies the histologic findings in a number of veins which had minimal to mild, with occasional moderate, degrees of hemorrhage in the peri-vascular area of the vein and in the adjacent subcutaneous connective tissue. These findings were judged most likely not due to drug effect or of a catheter having been present and are considered incidental.

FIG. 11 is a photograph of a cross-section proximal to the catheter injection site into the right cephalic vein of dog 2504, an animal that received a 2 mg/kg dose of Diprivan as described in Example 6. This was the only specimen in the study described in Example 6 that histologic examination found a lesion in a vein. Specifically, Animal 2504 had a focal area with a mild infiltrate of mononuclear cells on the endothelium that extended into the sub-endothelium and into the wall almost to the adventitia.

### DETAILED DESCRIPTION OF THE INVENTION

The terms “long-chain triglyceride” (LCT) or medium-chain triglycerides (MCT) are intended to have the following meanings. A LCT oil is a plant-derived oil composed of glycerol esters of fatty acids having a chain length of 14 to 22 carbons. The fatty acids in a LCT include both saturated fatty acids and unsaturated fatty acids. Because the oil is obtained from plants, the fatty acid composition of an LCT varies. A MCT oil is a synthetic oil which is obtained by hydrolysis of the fixed oil extracted from the hard, dried fraction of the endosperm of Cocos nucifera L., followed by re-esterification of the purified fatty acids with glycerol to produce MCT oils that are mainly glycerol esters of fatty acids having a chain length of six to twelve carbons including hexanoic or caproic acid (6 carbons), octanoate or caprylic acid (eight carbons), decanoic or capric acid (10 carbons), and dodecanoic or lauric acid (12 carbons) in a ratio of approximately 2:55:42:1.

The term “monoester” refers to a saturated alkyl ester of a saturated, monocarboxylic acid.

In the description of the invention that follows the composition is described as the percentage by weight of each ingredient relative to the weight of the complete emulsion. Thus, a text which reads “10% Y” means that the amount of the ingredient Y was 10 percent by weight relative to the weight of the total emulsion.

### A. Ingredients:

The ingredients used in compositions of the present invention are described here. Common ingredients such as water for injection or sodium hydroxide solution are not described.

Lipophilic Drug:
A therapeutic compound having a solubility in water of less than about 1 mg/mL. Preferably the lipophilic drug will comply with the specifications cited in the U.S. Pharmacopoeia (USP), the European Pharmacopoeia (Ph.Eur.), or another pharmacopoeia, although other specifications can be used. An example of a lipophilic drug of the invention is propofol, a drug also known as 2,6-bis(1-methylethyl)phenol or 2,6-diisopropylphenol. Propofol is identified by Chemical Abstracts Service (CAS) Registration Number 2078-54-8; and has a molecular formula C₁₂H₁₈O; and a molecular weight of 178.273. The content of propofol is 0.1-2% w/v of the composition, preferably 0.5-2% w/v, more preferably about 1-2% w/v and most preferably about 1% w/v of the emulsion composition. Other examples of poorly water-soluble, lipophilic drugs include: cancer chemotherapeutic agents (oncology drugs) such as doxorubicin, camptothecin, paclitaxel, etoposide, and daunorubicin; antibiotics such as the macrolides (e.g., erythromycin, solithromycin, azithromycin, and so forth); antifungal therapies such as the amphotericin B's, voriconazole, and posaconazole; immunotherapeutic agents such as cyclosporine, muramyl peptides, rapamycin, levamisole, and vaccines; and immuno-adjuvants such as squalene.

Primary Oil:
The primary oil is physiologically compatible, biocompatible, long-chain triglyceride oil selected from the group known as vegetable oils. Vegetable oils include, by way of example, almond oil, apricot kernel oil, avocado oil, canola oil, coconut oil, corn oil, cottonseed oil, flaxseed oil, grape seed oil, hazelnut oil, mustard oil, oat oil, olive oil, palm oil, peanut oil, rice bran oil, safflower oil, sesame oil, soybean oil, and sunflower oil. The primary oil used in compositions of the present invention is preferably refined, bleached, deodorized and preferably free of heavy metal contaminants. Soybean oil or olive oil is the preferred vegetable oil used in compositions of the present invention. Soybean oil or olive oil complying with specifications of the European Pharmacopoeia (Ph.Eur.) or the United States Pharmacopoeia (USP) is preferred.

Secondary Oil:
The secondary oil is a physiological, biocompatible ester of a saturated, monocarboxylic acid having a chain length of 4 to 10 carbons. The ester is selected from the group consisting of ethyl, butyl, and hexyl esters of saturated, monocarboxylic acids, wherein the monocarboxylic acid has from 4 to 8 carbons. In the alternative, the ester is selected from the group consisting of the esters of acetic acid (i.e., acetates) in which the alcohol residue is a saturated alkyl group having from 4 to 8 carbons. Properties of examples of secondary oils of the present invention are listed in Table 2.

TABLE 2
Examples of Secondary Oils of the Present Invention

| | | | | | | | Water |
|---|---|---|---|---|---|---|---|
| | No. of | | | | Vapor | Solubility | Solubility in |
| Name | Carbons | MW | Density | Log P | Pressure | in Water | Solvent |
| Butyl acetate | 6 | 116.2 | 0.88 | 1.8 | 10 mmHg | 0.64 wt % | 0.98 wt % |
| Hexyl | 8 | 144.2 | 0.87 | 2.4 | 1.3 mmHg | 0.043 wt % | 0.566 wt % |
| acetate | | | | | | | |
| Octyl acetate | 10 | 172.3 | 0.87 | 3.4 | 0.4 mmHg | 0.018 wt % | 0.02 wt % |
| Ethyl | 6 | 116.2 | 0.88 | 1.7 | 15.5 mmHg | 0.69 wt % | 0.79 wt % |
| butyrate | | | | | | | |
| Ethyl | 8 | 144.2 | 0.87 | 2.4 | 1.3 mmHg | 0.083 wt % | 0.47 wt % |
| hexanoate | | | | | | | |
| Ethyl | 10 | 172.3 | 0.87 | 3.5 | | 0.051 wt % | 0.90 wt % |
| octanoate | | | | | | | |

The secondary oil is selected to increase the solubility of the lipophilic drug in the oil phase and minimize the solubility of the lipophilic drug in the aqueous phase of the emulsion. By way of example, examination of the partition coefficients of propofol between the oil phase and the aqueous phase was used to select the secondary oil for propofol-containing emulsions of the invention. Partition coefficients typical of oils of the present invention are tabulated below (Table 3).

TABLE 3
Partition Coefficients of Propofol in
Exemplary Oils of the Present Invention

| Oil | Partition Coefficient |
|---|---|
| Ethyl butyrate | 19285 |
| Soybean oil | 6417 |
| Olive oil | 5720 |

The secondary oil and the primary oil are used in volumes that are mutually soluble one with the other and form a single, stable oil phase in the emulsion. The oil phase in the emulsion contains the bulk of the drug.
The secondary oil also serves as a bridging medium. Because the secondary oil is slightly soluble in water and water is slightly soluble in the secondary oil (Table 2), the secondary oil is capable of partially dissolving in the aqueous phase, where it can dissolve nanoparticles of the lipophilic drug that are present in the aqueous phase. Upon redissolution of the secondary oil in the oil phase of the emulsion, trace quantities of drug that were present in the aqueous phase as nanoparticles are transferred into the oil phase as solutes.

Emulsifying Agents:
In the oil-in water emulsion compositions of the present invention purified natural and/or synthetic agents are used as emulsifiers for stabilization of the oil-in-water emulsion. An ionic surfactant is used to provide repulsive force on the surface of the oil globules sufficient to minimize collisions that enhance coalescence into larger oil globules. The ionic surfactant is selected to ensure that the surfactant has minimal solubility in both water and oil; thus, it will mostly partition at the interface between the oil phase and the aqueous phase. Its concentration is chosen to ensure that the Debye length is comparable to the desired spacing between the oil droplets. An ionic surfactant of the present invention is selected from the group consisting of sodium myristate, sodium palmitate, sodium palmitoleate, sodium stearate, sodium oleate, sodium linoleate, sodium arachidate, and sodium behenate. A preferred ionic surfactant is sodium stearate, which is used in a concentration in the range of 0.001% to 0.00001%. In addition, a non-ionic surfactant is chosen to increase the rigidity of the interface between the oil phase and the aqueous phase and to stabilize against collisions that enhance the risk of coalescence into larger oil globules. The concentration of non-ionic surfactant is selected to maximize oil particle stability while avoiding introduction of adverse physiological effects associated with high concentrations of non-ionic surfactants. A concentration from about of 0.05% to about 3% is useful. Biocompatible, physiologically compatible surfactants that are already used in intravenous (IV) formulations are preferred, although novel surfactants may be employed. Examples of nonionic surfactants include poloxamers (amphiphilic block copolymers having relatively low polydispersity) which have hydroxyl, carboxylate, sulfate, ester, sugar, or amino end-groups.

Tonicity Modifying Agents:
The composition of the present invention is made isotonic to blood by incorporating a suitable tonicity modifying agent such as glycerin (glycerol), dextrose (D-glucose), or mannitol. Glycerin is the preferred tonicity modifying agent.

An intravenously administrable composition of the invention has a pH of 5-8.5 and preferably a pH of 6-8.5, conveniently adjusted by the presence of a relevant amount of aqueous acid (for example, hydrochloric acid) or aqueous alkali base (for example, sodium hydroxide).

### B. Emulsion Compositions of the Invention:

The composition of the lipophilic drug-containing oil-in-water emulsion comprises components listed in Table 4. It should be noted that the quantities of lipophilic drug, oil, emulsifiers, and tonicity agent may be varied independently within the limits disclosed below. Some useful variations in ingredients of emulsion compositions of the present invention are disclosed in Tables 5 and 6.

TABLE 4
Compositions of Emulsions of the Present Invention

| Formulation | | Concentration Range (all |
|---|---|---|
| Component | Example | percentages by weight) |
| Primary Oil | Soybean oil | 3-7% |
| | Olive oil | |
| Secondary Oil | Ethyl butyrate | 3-7% |
| Lipophilic Drug | Propofol | 0.5-5% |
| Tonicity Agent | Glycerol | 2% to 3% |
| Non-ionic Surfactant | Pluronic F-68 | 1-3% |
| Ionic Surfactant | Sodium stearate | 0.00005-0.0002% |
| Water | Sterile water for | As needed to total 100% |
| | injection | |

### C. Example Composition of the Formulation:

The composition of a propofol-containing oil-in-water emulsion comprises components listed in Table 5. It should be noted that the quantities of propofol, oil, emulsifiers, and tonicity agent may be varied independently within the limits disclosed below. Some useful variations in ingredients of emulsion compositions of the present invention are disclosed in Tables 6 and 7.

TABLE 5
Compositions of Emulsions of the Present Invention

| Formulation | | Concentration Range (all |
|---|---|---|
| Component | Example | percentages by weight) |
| Primary Oil | Soybean oil | 4-6% |
| | Olive oil | |
| Secondary Oil | Ethyl butyrate | 4-6% |
| Lipophilic Drug | Propofol | 0.5-2% |
| Tonicity Agent | Glycerol | 2% to 3% |
| Non-ionic Surfactant | Pluronic F-68 | 1-3% |
| Ionic Surfactant | Sodium stearate | 0.00005-0.0002% |
| Water | Sterile water for | As needed to total 100% |
| | injection | |

TABLE 6
Preferred Compositions of the Present Invention

| Formulation | | Concentration Range (All |
|---|---|---|
| Component | Example | percentages by weight) |
| Primary Oil | Soybean oil | 5% |
| Secondary Oil | Ethyl butyrate | 5% |
| Lipophilic Drug | Propofol | 1% |
| Tonicity Agent | Glycerol | 2.5% |
| Non-ionic Surfactant | Pluronic F-68 | 2% |
| Ionic Surfactant | Sodium stearate | 0.0001% |
| Water | Sterile water for | As needed to total 100% |
| | injection | |
| Particle Size | | Average oil particle diameter |
| | | less than 400 nm; |
| | | PFAT5 (number of particles |
| | | having an average diameter |
| | | of greater than 5 microns or |
| | | 5000 nm): Less than 0.05% |
| Osmolality | | 350-400 mOsm/kg |
| Bioburden | Presence of | Units are sterile and |
| | micro-organisms | endotoxin free |
| | Presence of | Less than 2 CFU of micro- |
| | endotoxin | organisms present |
| | | Less than 5.00 EU/mL |
| | | (endotoxin units/mL) |
| Free propofol | | Less than 12 microgram/mL |

TABLE 7
Composition of a Most Preferred Emulsion of the Present Invention

| Formulation | | Concentration Range (All |
|---|---|---|
| Component | Example | percentages by weight) |
| Primary Oil | Olive oil | 5% |
| Secondary Oil | Ethyl butyrate | 5% |
| Lipophilic Drug | Propofol | 1% |
| Tonicity Agent | Glycerol | 2.5% |
| Non-ionic Surfactant | Pluronic F-68 | 2% |
| Ionic Surfactant | Sodium stearate | 0.0001% |
| Water | Sterile water for | As needed to total 100% |
| | injection | |
| Particle Size | | Average particle size less |
| | | than 300 nm; |
| | | PFAT5 (number of particles |
| | | having an average diameter |
| | | of 5 microns): less than |
| | | 0.05% |
| Osmolality | | 350-400 mOsm/kg |
| Bioburden | Presence of | Units are sterile |
| | micro-organisms | Less than 2 CFU of micro- |
| | Presence of | organisms present |
| | endotoxin | Less than 5 EU/mL |
| | | (endotoxin units/mL) |
| Free propofol | | Less than 12 microgram/mL |

### D. the Process of Preparing an Emulsion Composition of the Present Invention:

The process of preparing a stable, intravenously administrable, propofol-containing, oil-in-water emulsion composition of the present invention employs conventional pharmaceutical manufacturing equipment and conventional processing steps. In order to avoid oxidation and/or degradation of a lipophilic drug during manufacture, the use of glass-lined manufacturing vessels and an inert atmosphere is preferred. In a process of the present invention, the following steps are employed. Each reagent is qualified for use in pharmaceutical manufacturing and is then accurately weighed and/or volumetrically transferred into mixing vessels having a composition appropriate for preparation of lipophilic drug-containing formulations.
1. Dissolution of the lipophilic drug in the oil phase. The requisite mass of a lipophilic drug is added to the requisite mass of either the primary oil or the secondary oil. The materials are agitated until a homogeneous solution is obtained. The requisite mass of the other oil is then added to the vessel and agitation is continued until a homogeneous solution is obtained.
2. Preparation of stock solutions of the emulsification agents. Stock solutions of each emulsification agent are prepared in sterile water. Each stock solution is prepared at a concentration at least 100-fold greater than the concentration required for the emulsion formulation to enable accurate transfer of the requisite quantity of the emulsification agent. Moderate heating and agitation are employed as needed to facilitate dissolution of each emulsification agent.
3. Preparation of the aqueous phase. The requisite mass of tonicity agent is added to a volume of sterile water equal to about 90% of the final volume of water required in the formulation. Sterile water is added in a volume needed to reach 100% of the final volume of water in the formulation. Measured portions of each emulsification agent are added in the volumes required to meet the specifications of the final formulation.
4. Mixing and coarse emulsification. The solution of the lipophilic drug in oil is added to the aqueous solution, and the mixture is agitated. A coarse homogenization is carried out with cooling as needed to prevent degradation and/or oxidation of sensitive formulation components. The mixture very rapidly turns milky white and expands slightly in volume due to the formation of bubbles at the surface.
5. Emulsification. A second homogenization is used to obtain an emulsion having the desired oil globule mean particle size in the range 80-300 nm and an acceptable particle size distribution. The mixture has a uniform milky white appearance.
6. Final formulation. If needed, the emulsion is gently agitated as small portions of dilute aqueous base or dilute aqueous acid are added to adjust the solution pH to a range of pH 5.0 to pH 8.5.
7. Aliquots having a volume equal to the target fill volume are transferred aseptically into clean, dry glass vials. After fill, the vials are closed and sealed with perfluoroethylene-coated, rubber-lined crimp caps.
8. Optionally, the closed and sealed, filled vials may be sterilized by autoclaving.

### Rationale for the Propofol-Containing Emulsion Compositions of the Present Invention

In the past, numerous emulsions containing 1% propofol in 10% oil have been prepared. After completion of a half-decade of research, the inventors have unexpectedly discovered that 10% oil-in-water emulsions containing 1% propofol in a combination of primary and secondary oils cause less injection-site pain, inflammation, and irritation than do conventional propofol emulsion compositions. Useful oils of the invention have the following characteristics:
Propofol is significantly more soluble in one oil than the other.
One oil is a long-chain triglyceride of natural origin.
The second oil is an ester of a saturated, monocarboxylic acid, wherein the ester contains a total of 6 to 12 carbon atoms.
One oil, the oil in which propofol is the more soluble, is more soluble in water than the other oil.
The solubility of water in one oil, the oil in which propofol is the more soluble, is greater than the solubility of water in the other oil.
The two oils are fully miscible.
The two oils form stable emulsions.

By way of example, the inventors unexpectedly discovered that emulsions made up of two oils, one of which was ethyl butyrate and the other of which was soybean oil or olive oil, reduced the concentration of “free” propofol in the aqueous phase and maximized its solubility in the oil phase. Data from a small study in beagle dogs indicated that the novel emulsion induced anesthesia with pharmacokinetics essentially identical to the pharmacokinetics of propofol administered as conventional emulsions but caused less injection site pain, inflammation, and tissue irritation than was observed following administration of conventional propofol emulsions. These data supported the widely accepted hypothesis that a reduced free propofol concentration in the aqueous phase of an oil-in-water emulsion resulted in decreased adverse effects such as tissue irritation, tissue and cellular inflammation, and pain at the injection site. Emulsions of the invention were stable during storage at 25° C. and 60% relative humidity for a period of at least about a year.

The inventors believe that it is chemically reasonable to distinguish an emulsion of their invention from a conventional propofol-containing emulsion using the following theory based on differential solubility. In conventional 1% propofol emulsions (e.g., Diprivan®), propofol partitions between the oil phase and the aqueous phase during emulsification. The bulk of propofol is dissolved in the oil phase of the emulsion, and a low concentration of propofol (“free” propofol) remains in the aqueous phase, where it associates into and is stabilized as tiny propofol nanodroplets suspended in the aqueous phase. In these conventional emulsions, there is no mechanism to facilitate transfer of the free propofol (or some fraction of it) from the aqueous phase to the oil phase.

In contrast, the inventors postulate that in emulsions of the present invention, several differential solubility mechanisms interact to maximize propofol solubility in the oil phase and minimize free propofol in the aqueous phase. The inventors developed this theory after comparison of the properties of a secondary oil of the invention, ethyl butyrate, to the properties of a primary oil of the invention, such as soybean oil or olive oil. Ethyl butyrate exhibits the following distinctive properties:
1. It is a significantly better solvent for propofol than either soybean or olive oil.
2. It is miscible with either soybean or olive oil.
3. It has low solubility in water.
4. Water has low solubility in ethyl butyrate.
5. It forms stable emulsions when emulsified with a second, emulsion-forming oil.

According to the inventors' theory, when an oil phase is made up of both a primary oil in which propofol has moderate solubility and a secondary oil in which propofol has greater solubility and the oil phase is emulsified with water, propofol will partition between the oil phase and the aqueous phase, as it does in conventional propofol-containing emulsions. However, emulsions of the invention contain an oil component which has low water solubility, e.g., ethyl butyrate. As the emulsion forms, the fraction of this oil component (e.g., ethyl butyrate) that is soluble in water can interact with the free propofol nanodroplets in the aqueous phase, dissolving the free propofol and transferring it to the bulk oil phase. As a result, after emulsification, the resulting emulsion contains a higher concentration of propofol in the oil phase and a lower concentration of the drug in the aqueous phase. The reduction in concentration of free propofol is marked by a reduction in adverse effects at the injection site. If this mechanism applies, the secondary oil (e.g., ethyl butyrate) serves both as a component of the oil phase that enhances propofol solubility in the oil phase and as a “bridging” oil having aqueous solubility sufficient to mix with the aqueous phase, interact with free propofol in the aqueous phase, and facilitate its transfer into the bulk oil phase.

### Advantages of the Invention

Briefly summarized, lipophilic drug-containing, oil-in-water emulsion compositions of the present invention provide the drug in a manner that is pharmacokinetically equivalent to conventional emulsions of the drug. In contrast to conventional emulsions of the drug, lipophilic drug-containing, oil-in-water emulsion compositions of the present invention exhibit greater stability during storage than conventional emulsions of the drug and cause fewer adverse effects such as injection site pain, inflammation, and tissue irritation than do conventional emulsions of the drug.

By way of example, propofol-containing, oil-in-water emulsion compositions of the present invention provide the drug in a manner that is pharmacokinetically equivalent to conventional propofol emulsions. In contrast to conventional propofol emulsions, propofol-containing, oil-in-water emulsion compositions of the present invention exhibit greater stability during storage than conventional propofol emulsions and cause less injection site pain, inflammation, and tissue irritation than do conventional propofol emulsions.

### EXAMPLES

The invention will now be illustrated by way of Examples. The Examples are provided by way of illustration only and in no way restrict the scope of the invention.

In some of these examples, a conventional propofol emulsion, Diprivan® (1% propofol) Injectable Emulsion (“Diprivan®”), is used as a control article and its properties are compared to the properties exhibited by test emulsions. Diprivan® (1% propofol) Injectable Emulsion, USP is a sterile, nonpyrogenic emulsion containing 10 mg/mL of propofol. Diprivan® is suitable for intravenous administration. In addition to the active component, propofol, the formulation contains soybean oil (100 mg/mL), glycerol (22.5 mg/mL), egg lecithin (12 mg/mL); and disodium edetate (EDTA; 0.005%); sodium hydroxide is used to adjust pH. DIPRIVAN Injectable Emulsion, USP is isotonic and has a pH of 7 to 8.5. [See: http://dailymed.nlm.nih.gov/dailymed/drugInfo.cfm?setid=d1ae9e26-ffd6-43df-bbd6-869cdede6afe; accessed Dec. 12, 2014.]

### Example 1

### Evaluation of the Stability of Oil-in-Water Macroemulsions During Storage at Ambient Temperatures

### Background:

When first prepared, oil-in-water macroemulsions are opaque, white to off-white liquids. A stable macroemulsion maintains this appearance over time. An unstable macroemulsion gradually undergoes phase separation, a dynamic process resulting in the formation of an upper layer that is opaque and white to off-white (i.e., an oil layer that has “creamed”) and a lower layer that changes from its original opaque appearance to clear and nearly colorless. Phase separation processes are often described as “Ostwald ripening.”

### Purpose:

Visual evaluation of the stability of oil-in-water macroemulsions during storage at ambient temperatures.

### Emulsion Formulation:

The requisite quantity of propofol was combined with specified quantities of oils to be studied (Table 8). The resulting fluid was agitated until homogeneous. Separately, stock solutions of 0.1% ionic surfactant in water and 20% nonionic surfactant in water were prepared. Aliquots of the stock ionic and nonionic surfactant solutions were added to deionized water and the resulting solution was added to the oil mixture at the desired concentration. The resulting mixture of oil and aqueous phases was shaken. Finally, the mixture was cooled in a water bath and emulsified with a probe sonicator. Aliquots of each emulsion were transferred to labeled, clean, dry glass bottles and closed and sealed with a screw cap. The emulsions were stored at ambient temperatures for 110 days. Periodically, the appearance of each emulsion was evaluated and a photograph was taken. If a “cream” layer had formed, the height of the “cream” layer was measured in the image. Formation of a cream layer is unacceptable, since it indicates an emulsion is unstable.

TABLE 8
Stability of Experimental Emulsions

| Oil Component | |
|---|---|
| of Emulsion | Emulsion Stability |
| Soybean oil | Stable |
| Olive oil | Stable |
| Isopropyl myristate | Unstable |
| Isopropyl palmitate | Unstable |
| Octanoic acid | Unstable |
| Ethyl butyrate | Unstable |
| 50:50 Mixture Soybean oil/Ethyl butyrate | Stable |
| 50:50 Mixture Olive oil/Ethyl butyrate | Stable |

### Results:

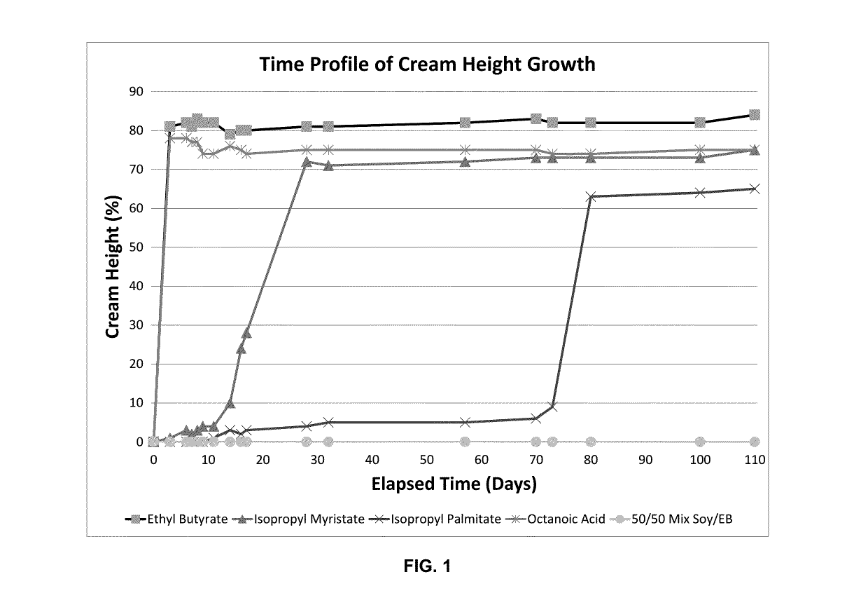

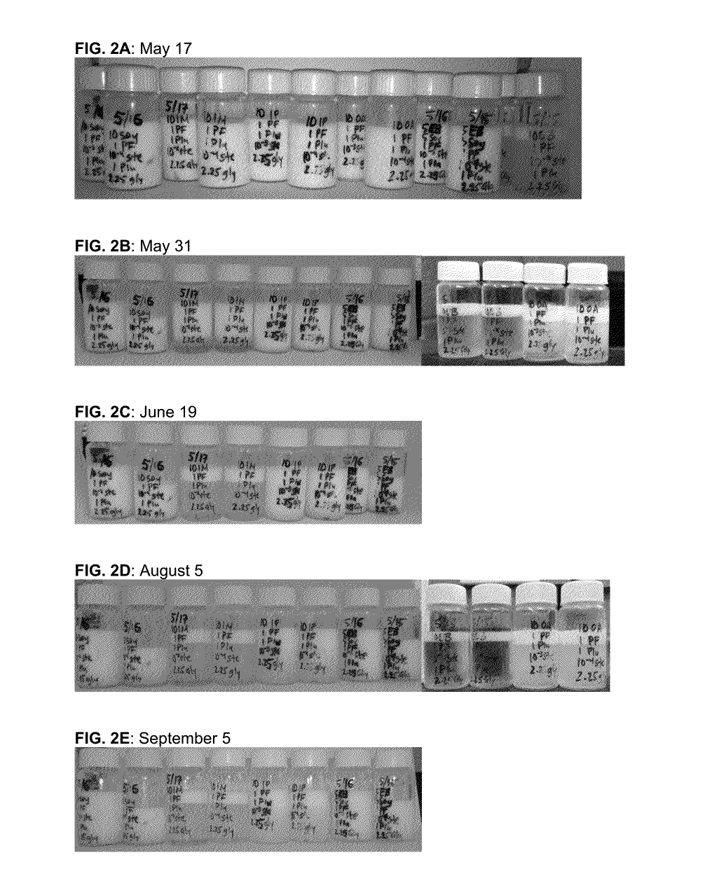

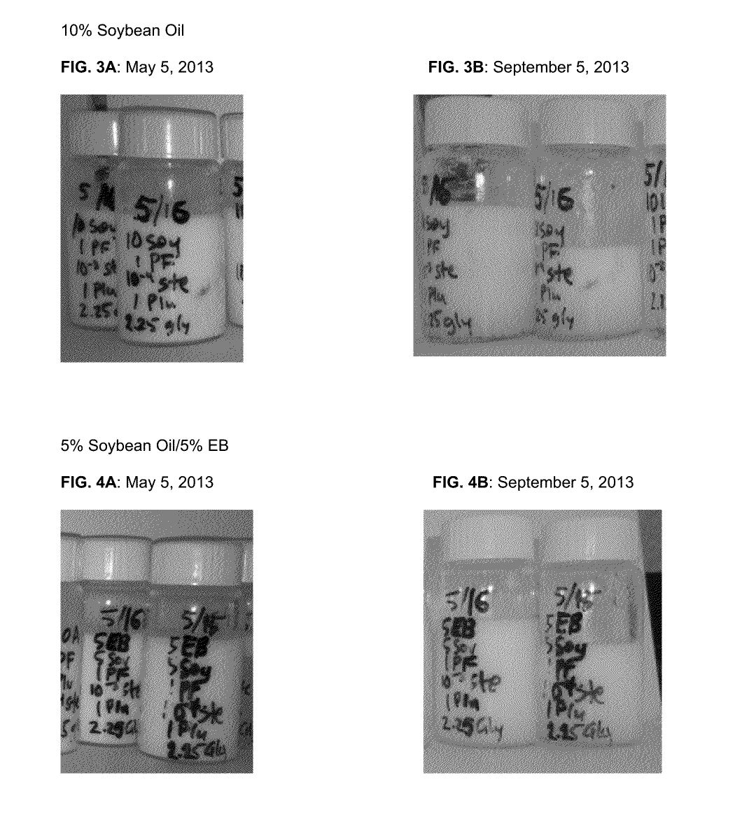

As summarized in Table 8 and shown in the graphic display in FIG. 1 and the photographs in FIG. 2, four of the eight trial formulations were stable during storage for 110 days at ambient temperatures. No creaming was observed in macroemulsions containing soybean oil, olive oil, a 50:50 mixture of soybean oil and ethyl butyrate, or a 50:50 mixture of olive oil and ethyl butyrate. In contrast, the other four formulations exhibited phase separation and creaming. Within days, macroemulsions containing ethyl butyrate or octanoic acid had separated into two distinct phases. Likewise, extensive creaming was observed in the isopropyl myristate emulsion after about 30 days of storage and in the isopropyl palmitate emulsion after about 80 days of storage. Unstable macroemulsions were not studied further.

### Example 2

### Evaluation of the Stability of Oil-in-Water Macroemulsions after Repeated Freeze-Thaw Cycles

### Background:

When first prepared, oil-in-water macroemulsions are opaque, nearly colorless liquids having a uniform average particle size. A stable macroemulsion maintains this average particle size after undergoing repetitive freezing and thawing (i.e., freeze-thaw cycles). An unstable macroemulsion exhibits changes in its average particle size as a result of this treatment.

### Purpose:

Changes in the average particle size of oil-in-water macroemulsions that had been subjected to freeze-thaw cycles were monitored in three successive experiments in which (a) the composition of the oil phase was changed (Experiment A); (b) the non-ionic surfactant was changed (Experiment B); and (c) the concentration of a preferred non-ionic surfactant (Pluronic F68) was changed (Experiment C).

### Experiment A: Effects of Changes in Composition of the Oil Phase Emulsion Formulation:

The requisite quantity of propofol was combined with specified quantities of oils to be studied (Table 9). The resulting fluid was agitated until homogeneous. Separately, stock solutions of 0.1% ionic surfactant in water and 20% nonionic surfactant in water were prepared. Aliquots of the stock ionic and nonionic surfactant solutions were added to deionized water and the resulting solution was added to the oil mixture at the desired concentration. The resulting mixture of oil and aqueous phases was shaken. Finally, the mixture was cooled in a water bath and emulsified with a probe sonicator. Aliquots of each emulsion were transferred to labeled, clean, dry glass bottles and closed and sealed with a screw cap.

TABLE 9
Oil Component of Emulsion

Soybean oil (“10 Soy”)
Olive oil (“10 Olive”)
50:50 Mixture Soybean oil/Ethyl butyrate (“5 Soy 5 EB”)
50:50 Mixture Olive oil/Ethyl butyrate (“5 Olive 5 EB”)

### Testing:

The four test emulsions and Diprivan (control formulation) were subjected to freeze-thaw cycles. After each cycle, an aliquot of the emulsion was removed and the average particle size was determined.

### Results:

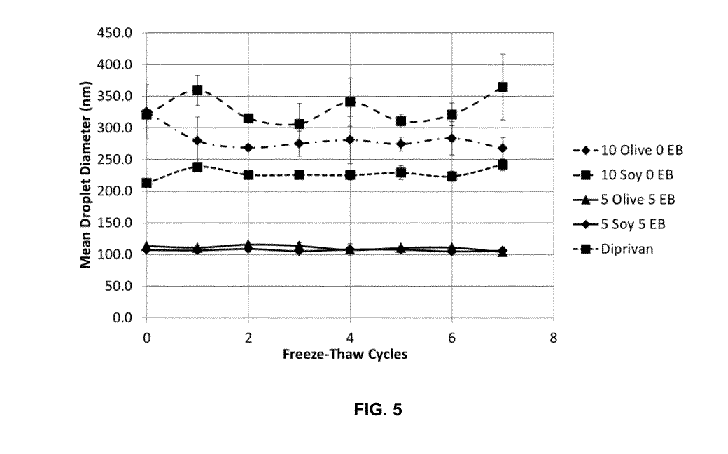

Experimental data are summarized in Table 10 and shown in FIG. 5.

TABLE 10
Changes in size of oil droplets as a result
of repetitive freeze-thaw cycling

| Oil Component | |
|---|---|
| of Emulsion | Result of Freeze-Thaw Cycling |
| Soybean oil | Average particle size fluctuated between 300 |
| | and 360 nm mean droplet diameter |
| Olive oil | Average particle size decreased from 325 nm |
| | mean droplet diameter to 275 nm mean droplet |
| | diameter and then remained relatively constant |
| 50:50 Mixture Soybean | Average particle size remained constant at |
| oil/Ethyl butyrate | about 110 nm mean droplet diameter |
| 50:50 Mixture Olive | Average particle size remained constant at |
| oil/Ethyl butyrate | about 110 nm mean droplet diameter |
| Diprivan | Average particle size increased from 210 nm |
| | mean droplet diameter to 240 nm mean droplet |
| | diameter and then remained relatively constant |

### Experiment B: Effects of Changes in the Non-Ionic Surfactant

### Emulsion Formulation:

The requisite quantity of propofol was combined with specified quantities of oils to be studied (Table 11). The resulting fluid was agitated until homogeneous. Separately, stock solutions of 0.1% ionic surfactant in water and 20% solutions in water of each of three nonionic surfactants were prepared. Aliquots of the stock ionic surfactant solution and one of the nonionic surfactant solutions were added to deionized water, and the resulting solution was added to the oil mixture at the desired concentration. The resulting mixture of oil and aqueous phases was shaken. Finally, the mixture was cooled in a water bath and emulsified with a probe sonicator. Aliquots of each emulsion were transferred to labeled, clean, dry glass bottles and closed and sealed with a screw cap.

TABLE 11
Composition of Experimental Emulsions of the Invention

| Oil Component | |
|---|---|
| of Emulsion | Nonionic Surfactant |
| 50:50 Mixture Soybean | Pluronic F68 |
| oil/Ethyl butyrate (“5 Soy 5 EB”) | |
| 50:50 Mixture Soybean | Tween 80 |
| oil/Ethyl butyrate (“5 Soy 5 EB”) | |
| 50:50 Mixture Soybean | Brij 78 |
| oil/Ethyl butyrate (“5 Soy 5 EB”) | |

### Testing:

The three test emulsions and Diprivan (control formulation) were subjected to freeze-thaw cycles. After each cycle, an aliquot of the emulsion was removed and the average particle size was determined.

### Results:

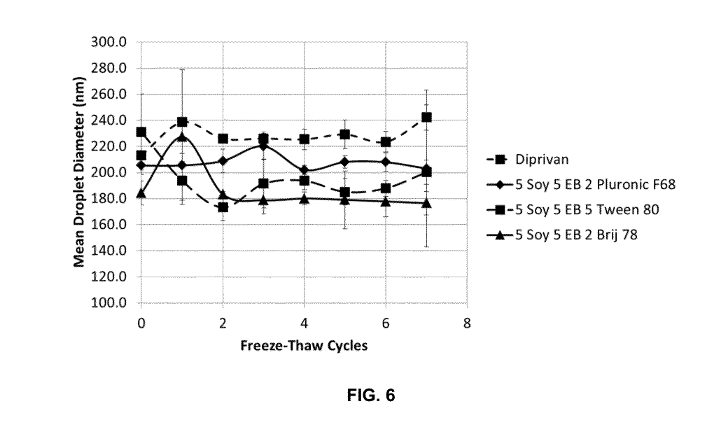

Experimental data are summarized in Table 12 and shown in FIG. 6. Analysis of these data indicated that Pluronic F68 was the nonionic surfactant of choice.

TABLE 12
Change in Mean Diameter of Oil Droplets as
a Result of Repetitive Freeze-Thaw Cycling

| Components | |
|---|---|
| of Emulsion | Result of Freeze-Thaw Cycling |
| 50:50 Mixture | Average particle size fluctuated between 200 |
| Soybean oil/Ethyl | and 200 nm mean droplet diameter |
| butyrate; | |
| Pluronic F68 | |
| 50:50 Mixture | Average particle size decreased from 325 nm mean |
| Soybean oil/Ethyl | droplet diameter to 275 nm mean droplet diameter |
| butyrate; | and then remained relatively constant within |
| Tween 80 | |
| 50:50 Mixture | Average particle size increased from about 230 nm |
| Soybean oil/Ethyl | mean droplet diameter to about 180 nm mean droplet |
| butyrate; | diameter and then remained relatively constant |
| Brij 78 | between 180 nm and 200 nm mean droplet diameter |
| Diprivan | Average particle size increased from about 220 nm |
| | mean droplet diameter to about 240 nm mean droplet |
| | diameter and then remained relatively constant |
| | within this range |

### Experiment C: Effects of Different Concentrations of Pluronic F68 on Emulsion Stability

### Emulsion Formulation:

The requisite quantity of propofol was combined with specified quantities of oils to be studied (Table 13). The resulting fluid was agitated until homogeneous. Separately, stock solutions of 0.1% ionic surfactant in water and 20% solutions in water of each of three concentrations of the nonionic surfactant Pluronic F68 were prepared. Aliquots of the stock ionic surfactant solution and one of the nonionic surfactant solutions were added to deionized water, and the resulting solution was added to the oil mixture at the desired concentration. The resulting mixture of oil and aqueous phases was shaken. Finally, the mixture was cooled in a water bath and emulsified with a probe sonicator. Aliquots of each emulsion were transferred to labeled, clean, dry glass bottles and closed and sealed with a screw cap.

TABLE 13
Composition of Experimental Emulsions of the Invention

| Oil Component | |
|---|---|
| of Emulsion | Nonionic Surfactant |
| 50:50 Mixture Soybean | 1% Pluronic F68 |
| oil/Ethyl butyrate (“5 Soy 5 EB”) | |
| 50:50 Mixture Soybean | 3% Pluronic F68 |
| oil/Ethyl butyrate (“5 Soy 5 EB”) | |
| 50:50 Mixture Olive | 1% Pluronic F68 |
| oil/Ethyl butyrate (“5 Olive 5 EB”) | |
| 50:50 Mixture Olive | 3% Pluronic F68 |
| oil/Ethyl butyrate (“5 Olive 5 EB”) | |

### Testing:

The four test emulsions and Diprivan (control formulation) were subjected to freeze-thaw cycles. After each cycle, an aliquot of the emulsion was removed and the average particle size was determined.

### Results:

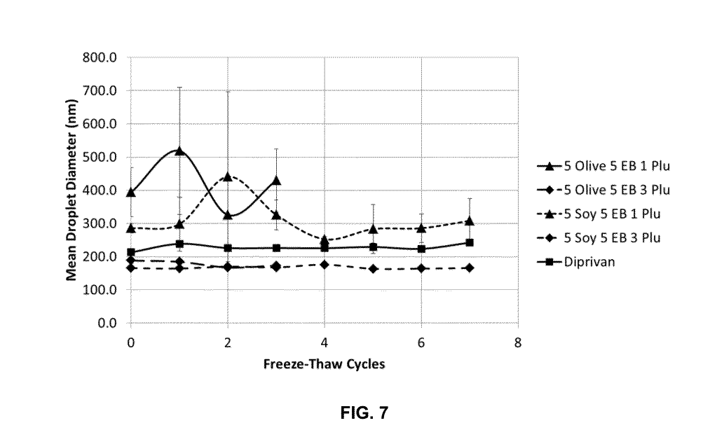

Experimental data are summarized in Table 14 and shown in FIG. 7. Analysis of these data indicated that emulsions containing a higher concentration of Pluronic F68 exhibited greater stability after repetitive freeze-thaw cycling.

TABLE 14
Change in Mean Diameter of Oil Droplets as
a Result of Repetitive Freeze-Thaw Cycling

| Components | |
|---|---|
| of Emulsion | Result of Freeze-Thaw Cycling |
| 50:50 Mixture | Average particle size increased from about 300 nm |
| Soybean oil/Ethyl | mean droplet diameter to about 450 nm mean droplet |
| butyrate; 1% | diameter and then decreased to the range from about |
| Pluronic F68 | 250 nm to about 300 nm mean droplet diameter |
| 50:50 Mixture | Average particle size remained relatively constant in |
| Soybean oil/Ethyl | the range from about 150 nm mean droplet diameter |
| butyrate; 3% | to about 180 nm mean droplet diameter |
| Pluronic F68 | |
| 50:50 Mixture | Average particle size increased from about 400 nm |
| Olive oil/Ethyl | mean droplet diameter to about 500 nm mean droplet |
| butyrate; 1% | diameter and then fluctuated between 320 nm and |
| Pluronic F68 | 425 nm mean droplet diameter |
| 50:50 Mixture | Average particle size remained relatively constant in |
| Olive oil/Ethyl | the range from about 150 nm mean droplet diameter |
| butyrate; 3% | to about 180 nm mean droplet diameter |
| Pluronic F68 | |
| Diprivan | Average particle size fluctuated from about 200 nm |
| | mean droplet diameter to about 250 nm mean droplet |
| | diameter |

### Example 3

### Evaluation of Changes in Composition of the Oil Phase on Propofol Concentration in the Aqueous Phase of Experimental Emulsions of the Invention

### Background:

The lipophilic drug propofol partitions between the oil phase and the aqueous phase of oil-in-water macroemulsions. Propofol that remains in the aqueous phase after emulsification is termed “free propofol.” Investigators have hypothesized that the concentration of free propofol in propofol-containing emulsions is directly related to the risk of pain and inflammation at the injection site. Therefore, an ideal propofol-containing emulsion will contain no measurable free propofol. Acceptable propofol-containing emulsions will contain free propofol at concentrations lower than those found in conventional propofol-containing emulsions (i.e., a concentration lower than about 12 micrograms/mL).

### Purpose:

To evaluate the free propofol concentration in 10% oil/1% propofol emulsions prepared from mixtures of primary oils with the secondary oil ethyl butyrate

### Emulsion Formulation:

The requisite quantity of propofol was combined with specified quantities of primary and secondary oils (Table 15). The primary oils that were studied included soybean oil (“soy”) and olive oil (“olive”). The secondary oil that was studied was ethyl butyrate (“EB”).

TABLE 15
Composition of the 10% Oil Phase of the Emulsions

| Primary Oil (%) | Secondary Oil (%) | Total Oil (%) |
|---|---|---|
| 100 | 0 | 100 |
| 80 | 20 | 100 |
| 60 | 40 | 100 |
| 40 | 60 | 100 |
| 20 | 80 | 100 |
| 0 | 100 | 100 |

The resulting fluid was agitated until homogeneous. Separately, stock solutions of 0.1% ionic surfactant in water and 20% nonionic surfactant in water were prepared. Aliquots of the stock ionic and nonionic surfactant solutions were added to deionized water and the resulting solution was added to the oil mixture at the desired concentration. The resulting mixture of oil and aqueous phases was shaken. Finally, the mixture was cooled in a water bath and emulsified with a probe sonicator.

### Methods for Determination of Free Propofol in the Aqueous Phase:

Two analytical approaches were used to measure free propofol in the aqueous phase. (A) A formulation identical to the emulsion formulation of interest was prepared without the surfactant. The mixture was stirred for a sufficient length of time to achieve equilibration. The mixture was then centrifuged, and the concentration of propofol in the aqueous phase was determined by HPLC. (B) The emulsion formulations were dialyzed against a 2.25% glycerol in water solution as the external phase. The dynamic concentration of propofol in the external phase was determined periodically using HPLC. The experiment was terminated when the propofol concentration exhibited no further changes (i.e., when equilibrium was achieved).

### HPLC Method:

Propofol exhibits a unique and strong absorbance peak at 270 nm, therefore this wavelength was used to determine propofol concentrations in unknown samples. The solvent used for HPLC was 50% acetonitrile and 50% water at a flow rate of 1 mL/min. The resulting operating pressure ranged between 3800-4000 psi. A Waters C18 column with a 4 μm internal diameter was used which eluted propofol between 3.9 and 4.5 minutes.

### Results:

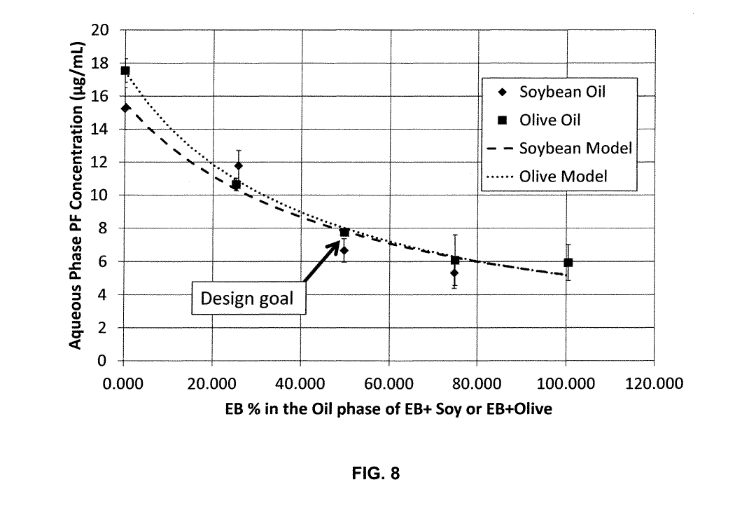

Experimental data are summarized in Table 16 and graphically illustrated in FIG. 8. The data show that emulsions met the acceptability criterion and had free propofol concentrations lower than those of conventional propofol formulations.

TABLE 16
Propofol Concentration (mg/L) in the Aqueous
Phase of Conventional Emulsions and Emulsions
of the Invention Containing 1% Propofol

| | Free Propofol | Standard |
|---|---|---|
| | Concentration | Deviation |
| Emulsion | (mg/L) | (mg/L) |
| A Conventional emulsion (Diprivan) | 11.7 | 2.0 |
| B (10% Soybean oil; 5% nonionic surfactant; | 10.7 | 0.2 |
| 10⁻⁴ wt. % ionic surfactant; 2.25% glycerol) | | |
| C (10% olive oil; 5% nonionic surfactant; | 12.9 | 2.9 |
| 10⁻⁴ wt. % ionic surfactant; 2.25% glycerol) | | |
| D (5% Soybean oil; 5% ethyl butyrate; 5% | 7.9 | |
| nonionic surfactant; 10⁻⁴ wt. % ionic | | |
| surfactant; 2.25% glycerol) | | |
| E (10% Olive oil; 5% ethyl butyrate; 5% | 7.9 | |
| nonionic surfactant; 10⁻⁴ wt. % ionic | | |
| surfactant; 2.25% glycerol) | | |

### Example 4

### Study of the Safety and Efficacy of Macrofol Formulations in Beagle Dogs

### Study Objectives:

In this non-GLP, exploratory study, 5/5/1 SBEB, 5/5/0 SBEB Excipient Control, 5/5/1 OOEB, or 5/5/0 OOEB Excipient Control were administered via 15 second (timed) fast bolus intravenous injection into the cephalic vein of a front leg of 2 male Beagle dogs.

### Test and Control Articles:

The test and control articles were supplied as closed and sealed, labeled vials that were opened under sterile technique immediately prior to use. Unused materials were discarded.

TABLE 17
Composition of the Test Articles

| | | Ethyl | | Olive | Sodium | | | | Particle |
|---|---|---|---|---|---|---|---|---|---|
| Emulsion | Propofol | Butyrate | Soybean | Oil | Stearate | Pluronic | Glycerol | Water | Size |
| Name | (%) | (%) | Oil (%) | (%) | (%) | F-68 (%) | (%) | (%) | (nm) |
| 5/5/0 | 0 | 4.98 | 5.00 | 0 | 0.000094 | 2.01 | 2.26 | 85.76 | 184 |
| SBEB | | | | | | | | | |
| 5/5/0 | 0 | 4.99 | 0 | 5.05 | 0.000097 | 2.02 | 2.34 | 85.61 | 223 |
| OOEB | | | | | | | | | |
| 5/5/1 | 0.99 | 5.00 | 4.97 | 0 | 0.000099 | 1.99 | 2.24 | 84.81 | 183 |
| SBEB | | | | | | | | | |
| 5/5/1 | 1.0 | 4.99 | 0 | 5.04 | 0.0001 | 1.99 | 2.26 | 84.72 | 232 |
| OOEB | | | | | | | | | |

Date of Manufacture: Nov. 1, 2013

### Animal Care, Selection & Treatment:

Animal welfare for this study was in compliance with the U.S. Department of Agriculture's (USDA) Animal Welfare Act (9 CFR Parts 1, 2, and 3), and the Guide for the Care and Use of Laboratory Animals. During acclimation and prior to selection for use in the study, the physical condition of each animal was evaluated by a veterinarian. The results of this assessment were used to assign each animal to category I or II per American Society of Anesthesiologists guidelines.

Animals selected for study were approximately 1.5 years-old young adults having body weights of approximately 12 kg. Through the duration of the study, dogs were group-housed in runs with elevated floors with >24 sq. ft. per kennel. Each kennel had an automatic watering device. Food (standard dog chow) was provided in steel feeders. The kennels conformed to standards set forth in the Guide for the Care and Use of Laboratory Animals. Feed was removed during the afternoon prior to dose and was not replaced until approximately 2 hours following dosing.

The body weight of each dog was determined pre-dose on dose days and at termination. On dosing days, dogs that experienced anesthetic effects were single-housed and closely monitored for health and well-being for approximately 2 to 4 hours post-dose, or until anesthetic effects were no longer evident. Then the dogs were group-housed as described above.

### Procedure:

The injection sites area on each front leg were shaved prior to the first dose. The needle poke-site of each dose was marked on the skin with permanent marker, as well as the location of the catheter tip. The marks were refreshed daily. 24-Gauge needle catheters were used. On Study Day 1, each dog was dosed with a total of two injections (one injection site per cephalic vein of each front leg). Doses were administered no less than 2 and no more than 4 hours apart at 10 mg/kg/dose, 0.2 mL/kg/dose. Sterile saline for injection was administered at 1 mL/kg into the catheter for 60 seconds (timed) beginning immediately following end of dosing. Catheter placement and formulation delivery were monitored by video recording to confirm adequacy of placement and drug delivery. Nose cone oxygen and pulse oximetry support for known anesthetic effects was provided after each dose in the groups that were expected to experience anesthetic effects, until the dog was awake and alert. Each applicable dog was bagged with oxygen during the procedure. An anesthesia machine with a ventilator (Narkomed; Draeger Medical, Inc., Telford, Pa.), and intubation equipment was maintained at the ready.

During injection and for 5 minutes thereafter, signs of respiratory distress and pain were documented. Induction characteristics and overall quality were assessed using a 3-point scale: 1. Smooth (without excitement); 2. Mild to moderate muscle twitching and limb movement; and 3. Very poor (severe excitement; muscle twitching, paddling of limbs; head movements and vocalization.

Injection sites and surrounding areas were observed as part of in-life clinical observations and scored using a modified Draize scoring system (Table 18).

TABLE 18
Modified Draize Scoring System for Skin
Irritation at the Injection Site

| Dermal | |
|---|---|
| Response | |
| Score | Observation |
| 0 | No evidence of irritation |
| 1 | Minimal erythema, barely perceptible |
| 2 | Definite erythema, readily visible; |
| | minimal edema or minimal papular response |
| 3 | Erythema and papules |
| 4 | Definite edema |
| 5 | Erythema, edema and papules |
| 6 | Vesicular eruption |
| 7 | Strong reaction spreading beyond the test site |

Out-of-cage clinical observations for general toxicity, moribundity, and mortality were conducted at least twice daily beginning immediately after dose on dose days. In addition, cage side clinical observations were conducted twice daily. Cage-side clinical assessments included, but were not limited to, assessment of activity, posture, respiration, hydration status, food consumption (qualitative), and overall body condition.

All dogs were euthanized approximately 24 hours after the last dose. Following euthanasia, the injection sites were evaluated grossly. The entire length of the cephalic veins that were injected, including a section 1 inch prior to the first poke site mark and 1 inch after the last catheter tip mark, were excised approximately 1 inch wide with subcutaneous tissue and cephalic vessel intact and included in the histopathological sample. Also, a vein from the hind leg of the animal was excised and fixed. Tissues that were harvested were processed immediately for histopathology. Gross pathology at the injection sites was photographed if visible damage was observed. The fixed tissues were delivered to a Board Certified Pathologist for analysis of each needle poke-site, catheter tip site, and surrounding tissues.

### Results:

Briefly summarized, the macroemulsions that were tested were well tolerated during administration of either the test or control article. If the test article was administered, the animal experienced anesthesia for the expected period and recovered without ill effects. Following dosing, all animals were healthy and well until euthanized.

Specifically, the following results were obtained.

Observations During Administration of the Test and Control Articles:

During injection and for 5 minutes thereafter, no signs of respiratory distress and pain were documented. Induction characteristics and overall quality were assessed using a 3-point scale, as described above. Uniformly, a score of 1 (smooth administration without excitement) was recorded for each dog.

In-Life Observations Immediately Following Dosing:
All animals tolerated the treatment well. No animal exhibited signs of distress, became moribund or died. Nose cone oxygen and pulse oximetry support for known anesthetic effects was provided after each dose in the groups that experienced anesthetic effects until the dog was awake and alert.

In-Life Observations Following Recovery and Prior to Euthanasia:
Injection sites and surrounding areas were observed as part of in-life clinical observations and scored using a modified Draize scoring system as described above. Uniformly, the scores recorded for the injection sites and surrounding areas of each animal were 0 (no evidence of irritation) or 1 (minimal or barely perceptible erythema). Out-of cage clinical observations for general toxicity, moribundity, and mortality were conducted at least twice daily beginning immediately after dose on dose days. No animal exhibited signs of general toxicity, moribundity, or mortality. In addition, cage side clinical observations were conducted twice daily. Cage-side clinical assessments included, but were not limited to, assessment of activity, posture, respiration, hydration status, food consumption (qualitative), and overall body condition. Each animal was deemed to exhibit age-appropriate activity, posture, respiration, hydration status, food consumption, and overall body condition.

Post-Euthanasia Observations and Histopathology:
Although major organs and tissues were removed from each animal and stored appropriately for histopathological examination, the study director saw no gross pathologies in the major organs and tissues of any animal at the time of excision and no subsequent histopathological evaluations of these organs and tissues were performed. The results of histopathological evaluation of dog veins are presented in Table 19.

TABLE 19
Results of Histopathological Evaluation of Dog Veins

| | | | Signs of | Signs of | Signs of | Other |
|---|---|---|---|---|---|---|
| Treatment | Tissue | Slide | Hemorrhage | Cellulitis | Vasculitis | Findings |
| 5/5/0 SBEB | Left | 1 | 2 perivascular | 0 | 0 | |
| | Puncture | | | | | |
| | Site | | | | | |
| | Left | 2 | 1 perivascular | 0 | 0 | |
| | Catheter | | | | | |
| | Tip | | | | | |
| 5/5/1 SBEB | Right | 3 | 0 | 0 | 0 | |
| | Puncture | | | | | |
| | Site | | | | | |
| | Right | 4 | 0 | 0 | C/A-1: focal | Swollen |
| | Catheter | | | | & very | endothelial |
| | Tip | | | | minimal | cells - focal & |
| | | | | | | very minimal |
| 5/5/0 OOEB | Left | 5 | 2 perivascular | 0 | 0 | |
| | Puncture | | | | | |
| | Site | | | | | |
| | Left | 6 | 1 perivascular | 0 | 0 | |
| | Catheter | | | | | |
| | Tip | | | | | |
| 5/5/1 OOEB | Right | 7 | 0 | 0 | C/A-1: focal | Swollen |
| | Puncture | | | | & very | endothelial |
| | Site | | | | minimal | cells - focal & |
| | | | | | | very minimal |
| | Right | 8 | 0 | 0 | C/A-1: focal | Swollen |
| | Catheter | | | | & very | endothelial |
| | Tip | | | | minimal | cells - focal & |
| | | | | | | very minimal |

### Example 5

### Emulsion Stability

The batch of Macrofol that was used in the preclinical study described in Example 6 (below) was produced on May 6, 2014. Product from this batch was placed into controlled storage at 25° C. and periodically evaluated for almost two years. The data in Table 20 confirm the absence of change in particle size and distribution, indicating that the emulsion was stable.

TABLE 20
Nanoparticle Size Data Summary

| | Cumulant Results | | NNLS Results | |
|---|---|---|---|---|
| | Z-Average | | Peak of | Peak Width |
| Sample ID | (nm) | Pdl | Interest (nm) | (nm) |

Date of Testing: 30 Sep. 2014 (Malvern Zetasizer)

| | | | | |
|---|---|---|---|---|
| Vial 1 | 157.0 | 0.13 | 179.5 | 69.95 |
| Vial 2 | 157.3 | 0.14 | 181.4 | 74.89 |
| Vial 3 | 157.4 | 0.13 | 179.0 | 88.80 |

Date of Testing: 1 Apr. 2015 (Malvern Zetasizer)

| | | | | |
|---|---|---|---|---|
| Vial 4 | 156.5 | 0.13 | 182.2 | 74.26 |
| Vial 5 | 157.4 | 0.11 | 178.4 | 65.77 |
| Vial 6 | 155.9 | 0.13 | 176.7 | 67.49 |

Date of Testing: 16 Jan. 2016 (Nanosight Nanoparticle Tracking Analysis)

| | | |
|---|---|---|
| Vial 7 | 158 | 46 |

Chapter 729 of the United States Pharmacopeia specifies that lipid emulsions suitable for intravenous delivery must contain less than 0.05% of the volume-weighted percentage of fat particles having a diameter greater than or equal to 5 μm (i.e., the “PFAT5” specification). The size of the lipid droplets is critical: because of mechanical filtration, larger-size fat globules (i.e., fat particles >5 μm in diameter) can be trapped in the lungs. Therefore, the injectable lipid emulsions of the present invention were evaluated to determine compliance with this standard. The particle size data shown in Table 20 confirms that lipid emulsions of the present invention meet the PFAT5 specification and contain less than 0.05% of a volume-weighted percentage of fat particles having a diameter greater than or equal to 5 μm.

### Example 6

### Another Study of the Safety and Efficacy of Macrofol Formulations in Beagle Dogs

### Study Objectives:

In this non-GLP, exploratory study, doses of a 5/5/1 Macrofol formulation (Test Article) or Diprivan® (1% propofol) Injectable Emulsion (Fresenius Kabi USA, Lake Zurich, Ill.; Control Article) were administered via timed fast bolus intravenous injection into the cephalic vein of a front leg of male Beagle dogs.

### Test and Control Articles:

The Test Article had the composition described in Table 21. The Control Article was purchased from a commercial source. The Test and Control Articles were supplied as closed and sealed, labeled vials that were opened under sterile technique immediately prior to use. Unused materials were discarded.

TABLE 21
Composition of the Test Article

| | | Ethyl | | Sodium | | | | Average |
|---|---|---|---|---|---|---|---|---|
| Emulsion | Propofol | Butyrate | Olive Oil | Stearate | Pluronic | Glycerol | Water | Particle |
| Name | (%) | (%) | (%) | (%) | F-68 (%) | (%) | (%) | Size (nm) |
| 5/5/1 | 1.0 | 4.99 | 5.04 | 0.0001 | 1.99 | 2.26 | 84.72 | 157 |
| Macrofol | | | | | | | | |
| (“Macrofol”) | | | | | | | | |

Date of Manufacture: May 6, 2014

### Animal Care, Selection & Treatment:

Animal welfare for this study was in compliance with the U.S. Department of Agriculture's (USDA) Animal Welfare Act (9 CFR Parts 1, 2, and 3), and the Guide for the Care and Use of Laboratory Animals. During acclimation and prior to selection for use in the study, the physical condition of each animal was evaluated by a veterinarian. The results of this assessment were used to assign each animal to category I or II per American Society of Anesthesiologists guidelines.

Animals selected for study were young adult males having body weights of ranging from about 7 kg to about 12 kg. Through the duration of the study, dogs were group-housed in runs with elevated floors with >24 sq. ft. per kennel. Each kennel had an automatic watering device. Food (standard dog chow) was provided in steel feeders. The kennels conformed to standards set forth in the Guide for the Care and Use of Laboratory Animals. Feed was removed during the afternoon prior to dose and was not replaced until approximately 2 hours following dosing.

The body weight of each dog was determined pre-dose on dose days and at termination. On dosing days, dogs that experienced anesthetic effects were single-housed and closely monitored for health and well-being for approximately 2 to 4 hours post-dose, or until anesthetic effects were no longer evident. Then the dogs were group-housed as described above.

### Procedure:

Sixteen dogs were used in the study. Eight dogs were selected at random and assigned to Group 1 and received doses of 5/5/1 Macrofol formulation. Eight dogs were selected at random and assigned to Group 2 and received doses of Diprivan®.

The injection sites area on each front leg were shaved prior to the first dose. The needle poke-site of each dose was marked on the skin with permanent marker, as well as the location of the catheter tip. The marks were refreshed daily. 24-Gauge needle catheters were used.

On Day 1 each animal in Group 1 received a 2 mg/kg dose of the Macrofol formulation in the right foreleg. The dose was administered via intravenous infusion into a cephalic vein at a constant rate of 0.2 ml/kg via 15 second (timed) fast bolus which is equivalent to 2 mg/kg dosing. On Day 5 each animal in Group 1 received a 5 mg/kg dose of the Macrofol emulsion in the left foreleg. The dose was administered as a fast bolus at a constant rate of 0.5 ml/kg which was delivered over 30 seconds.

In like manner, on Day 1 each animal in Group 2 received a 2 mg/kg dose of Diprivan® (Fresenius Kabi, Lake Zurich, Ill.) in the right foreleg. The dose was administered via intravenous infusion into a cephalic vein at a constant rate of 0.2 ml/kg via 15 second (timed) fast bolus which is equivalent to the 2 mg/kg dosing. On Day 5 each animal in Group 2 received a 5 mg/kg dose of the Macrofol emulsion in the left foreleg. The dose was administered as a fast bolus at a constant rate of 0.5 ml/kg which was delivered over 30 seconds.

Sterile saline for injection was administered to each animal in both groups at 1 mL/kg into the catheter for 60 seconds (timed) beginning immediately following end of each dosing. Catheter placement and formulation delivery were monitored by video recording to confirm adequacy of placement and drug delivery. Nose cone oxygen and pulse oximetry support for known anesthetic effects was provided after each dose in the groups that were expected to experience anesthetic effects, until the dog was awake and alert. Each applicable dog was bagged with oxygen during the procedure. An anesthesia machine with a ventilator (Narkomed; Draeger Medical, Inc., Telford, Pa.), and intubation equipment was maintained at the ready.

During injection and for 5 minutes thereafter, signs of respiratory distress and pain were documented. Induction characteristics and overall quality were assessed using a 3-point scale: 1. Smooth (without excitement); 2. Mild to moderate muscle twitching and limb movement; and 3. Very poor (severe excitement; muscle twitching, paddling of limbs; head movements and vocalization.

On Day 1, blood for pharmacokinetic analysis was obtained from the jugular vein of 5 dogs in each group at the following times: approximately pre-dose, 1, 5, 10, 20 min, and 1 hour post-dose. Blood was collected into EDTA tubes. Blood was then centrifuged at approximately 3,200 RPM for 10 minutes in a refrigerated centrifuge and samples of plasma were obtained. Samples of plasma were frozen at −80° C. until 6 plasma samples each from 2 animals—Animal numbers #1002 (Macrofol) and 2002 (Diprivan), total 12 samples] were shipped to a qualified testing laboratory for HPLC analysis (propofol determination) and calculation of pharmacokinetic parameters.

Injection sites and surrounding areas were observed as part of in-life clinical observations and scored using a modified Draize scoring system (Table 22).

TABLE 22
Modified Draize Scoring System for Skin
Irritation at the Injection Site

| Dermal | |
|---|---|
| Response | |
| Score | Observation |
| 0 | No evidence of irritation |
| 1 | Minimal erythema, barely perceptible |
| 2 | Definite erythema, readily visible; |
| | minimal edema or minimal papular response |
| 3 | Erythema and papules |
| 4 | Definite edema |
| 5 | Erythema, edema and papules |
| 6 | Vesicular eruption |
| 7 | Strong reaction spreading beyond the test site |

Out-of cage clinical observations for general toxicity, moribundity, and mortality were conducted at least twice daily beginning immediately after dose on dose days. In addition, cage side clinical observations were conducted twice daily. Cage-side clinical assessments included, but were not limited to, assessment of activity, posture, respiration, hydration status, food consumption (qualitative), and overall body condition.

All dogs were euthanized approximately 24 hours after the last dose. Following euthanasia, the injection sites were evaluated grossly. The entire length of the cephalic veins that were injected, including a section 1 inch prior to the first poke site mark and 1 inch after the last catheter tip mark, were excised approximately 1 inch wide with subcutaneous tissue and cephalic vessel intact and included in the histopathological sample. Also, a vein from the hind leg of the animal was excised and fixed. Tissues that were harvested were processed immediately for histopathology. Gross pathology at the injection sites was photographed if visible damage was observed. The fixed tissues were delivered to a Board Certified Pathologist for analysis of each needle poke-site, catheter tip site, and surrounding tissues.

### Results:

Briefly summarized, the macroemulsions that were tested were well tolerated during administration of either the test or control article. If the Test Article was administered, the animal experienced anesthesia for the expected period and recovered without ill effects. If Diprivan® was administered at the 5 mg/kg dose, the animal experienced anesthesia for the expected period and recovered without ill effects. Following dosing, all animals were healthy and well until euthanized.

Specifically, the following results were obtained.

Observations During Administration of the Test and Control Articles:
During injection and for 5 minutes thereafter, no signs of respiratory distress and pain were documented. Anesthesia induction score, as determined by the 3-point scale described above, did not appear to be different between the Macrofol- and Diprivan®-treated groups dosed at 2 mg/kg or 5 mg/kg (1.1 vs. 1.0).

In-Life Observations Immediately Following Dosing:
All animals tolerated the treatment well. No animal exhibited signs of distress, became moribund or died. Nose cone oxygen and pulse oximetry support for known anesthetic effects was provided after each dose in the groups that experienced anesthetic effects until the dog was awake and alert. The time required for induction appeared to be similar in Macrofol-vs. Diprivan®-treated groups at both dose levels. The duration of anesthesia appeared to be longer in Macrofol-treated animals compared to those receiving Diprivan®, both at the 2 mg/kg dose and at the 5 mg/kg dose. No sedation was observed in seven of eight dogs treated with 2 mg/kg of Diprivan®, and only one dog experienced sedation for about 3 minutes. However, seven of eight dogs given 2 mg/kg of Macrofol achieved sedation with a group average of 7 minutes. Sedation was achieved in all dogs given 5 mg/kg of either drug with an average duration of sedation in dogs given Macrofol being longer; 21:11 minutes versus 7:53 minutes in dogs given Diprivan®.

In-Life Observations Following Recovery and Prior to Euthanasia:
Injection sites and surrounding areas were observed as part of in-life clinical observations and scored using a modified Draize scoring system as described above. Uniformly, the scores recorded for the injection sites and surrounding areas of each animal were 0 (no evidence of irritation) or 1 (minimal or barely perceptible erythema). No skin abnormalities were observed. Out-of cage clinical observations for general toxicity, moribundity, and mortality were conducted at least twice daily beginning immediately after dose on dose days. No animal exhibited signs of general toxicity, moribundity, or mortality. In addition, cage side clinical observations were conducted twice daily. Cage-side clinical assessments included, but were not limited to, assessment of activity, posture, respiration, hydration status, food consumption (qualitative), and overall body condition. Each animal was deemed to exhibit age-appropriate activity, posture, respiration, hydration status, food consumption, and overall body condition.

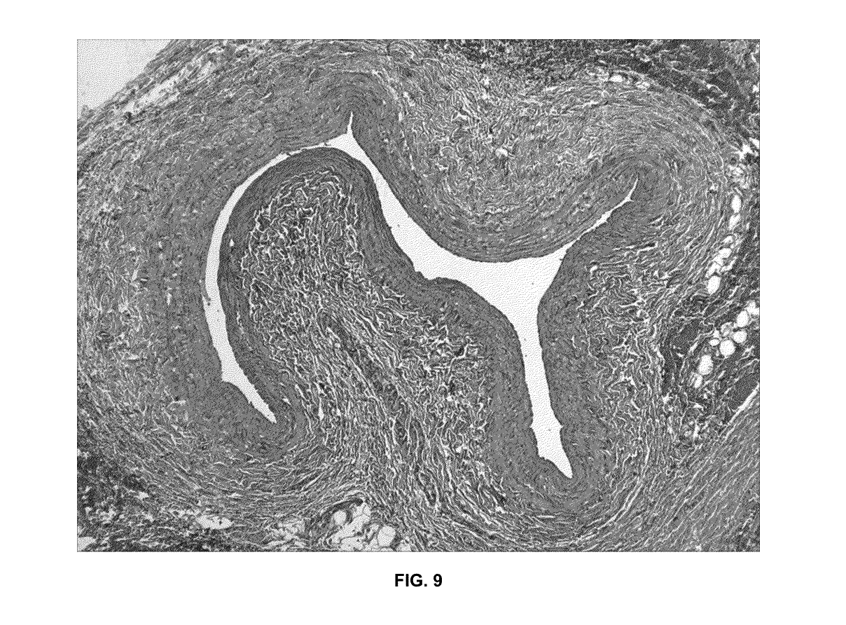

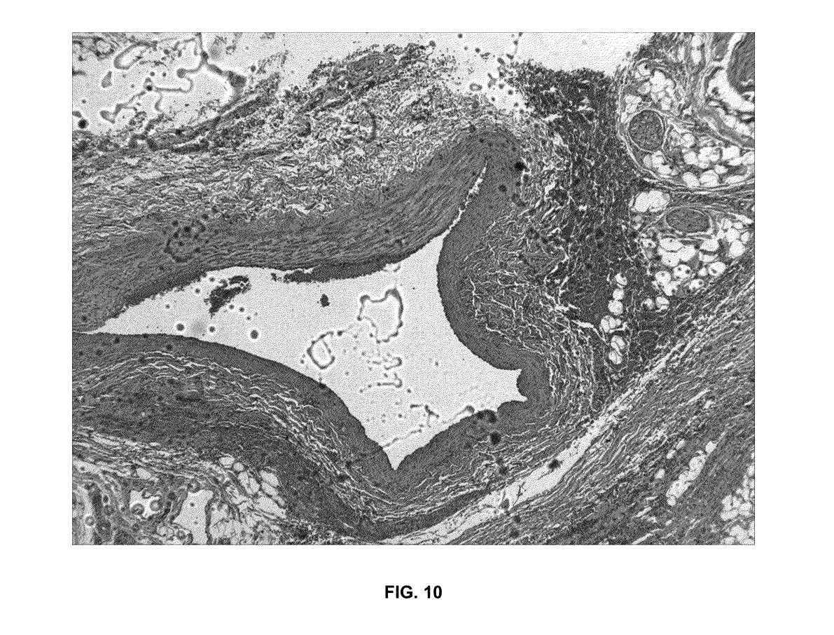

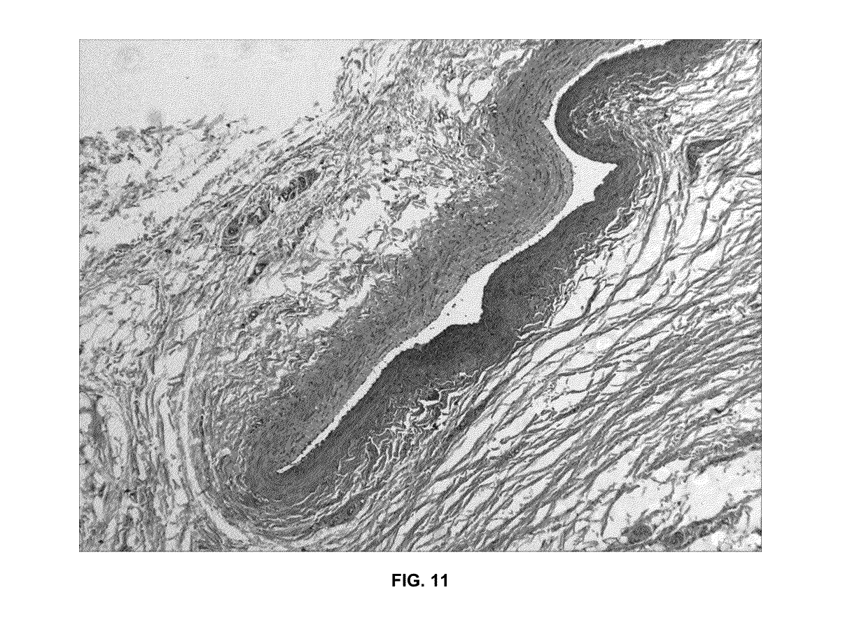

Post-Euthanasia Observations and Histopathology:
Although major organs and tissues were removed from each animal and stored appropriately for histopathological examination, the study director saw no gross pathologies in the major organs and tissues of any animal at the time of excision and no subsequent histopathological evaluations of these organs and tissues were performed.
Cross-sections of cephalic vein from the right and left forelegs of each animal were excised and prepared for analysis by transferring to glass slides and staining with hematoxylin and eosin. In addition, cross-sections of sephanous vein were excised from each animal and prepared for analysis in a similar manner. The cross-sections of sephanous vein served as control tissues. One hundred twelve glass slides with hematoxylin and eosin stained canine skin sections containing either a cross-section of cephalic vein (3 sections per leg) or sephanous vein (control tissue) were submitted for histopathologic evaluation. The tissues were evaluated without knowledge of the specific pharmacologic activity or formulation that was administered to the dog from which the tissues were derived. The results of histopathological evaluation of dog veins are presented in Table 23 and representative slides are shown in FIGS. 9-11.
FIG. 9 is a photograph of a cross-section of the left cephalic vein of dog 1002, an animal that received a 5 mg/kg dose of Macrofol. The section was proximal to the catheter tip. This photograph exemplifies the histologic findings of all slides from this study except for those shown in FIGS. 10 and 11. The findings were deemed unremarkable and reported as nonspecific findings (NSF).
FIG. 10 is a photograph of a cross-section of the left cephalic vein of dog 2503, an animal that received a 5 mg/kg dose of Diprivan. This photograph exemplifies the histologic findings in a number of veins which had minimal to mild, with occasional moderate, degrees of hemorrhage in the peri-vascular area of the vein and in the adjacent subcutaneous connective tissue. These findings were judged most likely not due to drug effect or of a catheter having been present and are considered incidental.
FIG. 11 is a photograph of a cross-section proximal to the catheter injection site into the left cephalic vein of dog 2504, an animal that received a 5 mg/kg dose of Diprivan. This was the only specimen in this study that histologic examination found a lesion in a vein. Specifically, this tissue from Animal 2504 had a focal area with a mild infiltrate of mononuclear cells on the endothelium that extended into the sub-endothelium and into the wall almost to the adventitia.

TABLE 23
Results of Histopathological Evaluation of Dog Cephalic Veins

| | ID | | Slide | Signs of | Signs of | Signs of | Other |
|---|---|---|---|---|---|---|---|
| Treatment | No. | Tissue | No. | Hemorrhage | Cellulitis | Vasculitis | Findings |
| Macrofol at | 1001 | Rt. | 1-2 | NSF | NSF | NSF | NSF |
| a dose of 2 mg/kg | | foreleg | | | | | |
| | 1002 | Rt. | 1-3 | NSF | NSF | NSF | NSF |
| | | foreleg | | | | | |
| | 1003 | Rt. | 1-3 | NSF | NSF | NSF | NSF |
| | | foreleg | | | | | |
| | 1004 | Rt. | 1-3 | NSF | NSF | NSF | NSF |
| | | foreleg | | | | | |
| | 1005 | Rt. | 1-3 | NSF | NSF | NSF | NSF |
| | | foreleg | | | | | |
| | 1006 | Rt. | 1-3 | NSF | NSF | NSF | NSF |
| | | foreleg | | | | | |
| | 1007 | Rt. | 1-3 | NSF | NSF | NSF | NSF |
| | | foreleg | | | | | |
| | 1008 | Rt. | 1-3 | NSF | NSF | NSF | NSF |
| | | foreleg | | | | | |
| Macrofol at | 1001 | Lft | 4-6 | NSF | NSF | NSF | NSF |
| a dose of 5 mg/kg | | foreleg | | | | | |
| | 1002 | Lft | 4-6 | NSF | NSF | NSF | NSF |
| | | foreleg | | | | | |
| | 1003 | Lft | 4-6 | NSF | NSF | NSF | NSF |
| | | foreleg | | | | | |
| | 1004 | Lft | 4-6 | NSF | NSF | NSF | NSF |
| | | foreleg | | | | | |
| | 1005 | Lft | 4-6 | NSF | NSF | NSF | NSF |
| | | foreleg | | | | | |
| | 1006 | Lft | 4-6 | NSF | NSF | NSF | NSF |
| | | foreleg | | | | | |
| | 1007 | Lft | 4-6 | NSF | NSF | NSF | NSF |
| | | foreleg | | | | | |
| | 1008 | Lft | 4-6 | NSF | NSF | NSF | NSF |
| | | foreleg | | | | | |
| Diprivan ® | 2001 | Rt. | 1-3 | NSF | NSF | NSF | NSF |
| at a dose of | | foreleg | | | | | |
| 2 mg/kg | 2002 | Rt. | 1-3 | NSF | NSF | NSF | NSF |
| | | foreleg | | | | | |
| | 2503 | Rt. | 1-3 | NSF | NSF | NSF | NSF |
| | | foreleg | | | | | |
| | 2504 | Rt. | 1-3 | NSF | NSF | NSF | NSF |
| | | foreleg | | | | | |
| | 2505 | Rt. | 1-3 | NSF | NSF | NSF | NSF |
| | | foreleg | | | | | |
| | 2506 | Rt. | 1-3 | NSF | NSF | NSF | NSF |
| | | foreleg | | | | | |
| | 2507 | Rt. | 1-3 | NSF | NSF | NSF | NSF |
| | | foreleg | | | | | |
| | 2508 | Rt. | 1-3 | NSF | NSF | NSF | NSF |
| | | foreleg | | | | | |
| Diprivan ® | 2001 | Lft | 4-6 | NSF | NSF | NSF | NSF |
| at a dose of | | foreleg | | | | | |
| 5 mg/kg | 2002 | Lft | 4-6 | NSF | NSF | NSF | NSF |
| | | foreleg | | | | | |
| | 2503 | Lft | 4-6 | NSF | NSF | NSF | NSF |
| | | foreleg | | | | | |
| | 2504 | Lft | 4-6 | NSF | NSF | NSF | Slide 4 |
| | | foreleg | | | | | showed mild |
| | | | | | | | inflammation |
| | 2505 | Lft | 4-6 | NSF | NSF | NSF | NSF |
| | | foreleg | | | | | |
| | 2506 | Lft | 4-6 | NSF | NSF | NSF | NSF |
| | | foreleg | | | | | |
| | 2507 | Lft | 4-6 | NSF | NSF | NSF | NSF |
| | | foreleg | | | | | |
| | 2508 | Lft | 4-6 | NSF | NSF | NSF | NSF |
| | | foreleg | | | | | |

* Rt = right; Lft = left; by “NSF” is meant that only nonspecific findings were observed.

### Example 7

### A Study of the Safety and Efficacy of Squalene Emulsion Formulations in Balb/c Mice

### Study Objectives:

In this non-GLP, exploratory study, doses of a 5/5/4 Macrofol-S formulation containing 4% squalene (Test Article) will be administered intramuscularly into Balb/c mice. The adjuventicity of the formulation will be evaluated in the context of RSV vaccination. RSV is a negative-stranded RNA virus that causes significant respiratory disease in infants, immune-compromised and older adults.

### Test Article:

The Test Article will have the composition described in Table 24. The Test Article will be supplied as closed and sealed, labeled vials that will be opened under sterile technique immediately prior to use. Unused materials will be discarded.

TABLE 24
Composition of the Test Article

| | | Octyl | | Sodium | | Sodium | | Average |
|---|---|---|---|---|---|---|---|---|
| Emulsion | Squalene | Acetate | Olive | Stearate | Polysorbate- | Chloride | Water | Particle |
| Name | (%) | (%) | Oil (%) | (%) | 80 (PS80; %) | (%) | (%) | Size (nm) |
| 5/5/4 | 4 | 4.99 | 5.04 | 0.0001 | 1.99 | 1 | 84.72 | 80 |
| Macrofol-S | | | | | | | | |
| (“Macrofol- | | | | | | | | |
| S”) | | | | | | | | |

### Control Article:

The Control Article will be the commercially available oil-in-water emulsion AddaVax™ (Invivogen, San Diego, Calif.). AddaVax has an average particle size of 160 nm. The Control Article will be supplied as closed and sealed, labeled vials that will be opened under sterile technique immediately prior to use. Unused materials will be discarded.

### Model Antigen:

RSV F will be used as a model antigen to test the immunogenicity of the Macrofol-S emulsion preparation. The fusion F protein of RSV A2 will expressed in Chinese Hamster Ovary (CHO) cells (ATCC, Manassas, Va.) to >95% purity.

### Animal Care, Selection & Treatment:

Animal welfare for this study will be in compliance with the guidelines of the Institutional Animal Care and Use Committee.

### Procedure:

Mice will be randomly assigned into 5 groups (n=5 mice per group), primed at day 0 and boosted at day 14 intramuscularly (i.m.) with 1.5 μg of RSV F and Macofol-S emulsions +/−20 μg of CpG 2395 (Invivogen, San Diego, Calif.) or with 1.5 μg of RSV F and AddaVax +/−20 μg of CpG 2395. (CpG 2395 is a toll-like receptor 9 agonist that detects danger signals and stimulates immune responses. The 1.5 μg dose is based on observations showing that RSV F+AddaVax/CpG 2395 immunization induces complete protection against RSV A2 challenge.) Mice will be challenged intranasally (i.n.) at day 28 with 106 pfu of RSV A2. The RSV infection control group will be infected at day 0 and challenged at day 28 i.n. with 106 pfu of RSV A2. All groups will be retro-orbitally bled at day 0 (6 h post-priming) and at day 28 before viral challenge. All animals will be euthanized at day 35. At that time blood will be harvested for serum IgG isotyping; spleens will be harvested for analysis of cellular immune responses; and lungs and nasal turbinates will be harvested for RSV A2 titer measurement by plaque assay.

### Micro-Neutralization Assay:

Serum samples at day 35 will be heat inactivated at 56° C. for 45 minutes. In 96-well plates the control antibody (Synagis®) will be serially diluted by 3-fold increments (starting at 8 μg/ml) in cell culture media (minimal essential medium (MEM) supplemented with 5% heat-inactivated fetal bovine serum (FBS), 2 mM L-glutamine, 100 U of penicillin/mL, and 100 μg of streptomycin/mL for a final volume of 50 μL. In duplicate, the test sera (starting dilution 1:2) will be serially diluted by 3-fold increments in cell culture media for a final volume of 50 μL. Each serum dilution will be mixed with 50 μL RSV A2 at 500 pfu per well. Following 2 hour incubation at 37° C. with 5% CO₂, 2.5×10⁴ HEp-2 cells in 100 μL volume will be added to each well. Cells plus virus and cell only wells will serve as controls. After 3 days of incubation at 37° C. with 5% CO₂, the cell culture medium will be removed and the monolayer will be fixed with chilled 80% acetone. RSV replication will be visualized by immunostaining with an HRP-labeled 1331H monoclonal antibody. The reciprocal log 2 of the 1050 will be determined for each serum sample using Prism GraphPad software.

### Serum IgG, IgG1, IgG2a ELISA:

RSV F-specific IgG antibody titers will be measured in serum at day 32. High binding 96-well plates will be coated with RSV F at 100 ng/well. Control antibodies (purified 1331H for total IgG and IgG2a and purified 1308F for IgG1 will be serially diluted by 3-fold increments starting from a concentration of 1 μg/mL in sample diluent (PBS with 1% BSA and 0.05% Tween 20). Samples will be diluted in sample diluent at 1:100 for non-immunized animals, at 1:10⁵ and 1:10⁶ for RSV A2 immunized animals and 1:10⁶ and 1:10⁷ for RSV F+adjuvant immunized animals. Bound total IgG, IgG1 or IgG2a will be detected with the appropriate HRP-labeled antibody (HRP-labeled goat anti-mouse IgG, HRP-labeled goat anti-mouse IgG1 antibody, HRP-labeled goat anti-mouse IgG2a antibody). The serum antibody titers will be calculated based on the standard curves to determine pg/ml of each antibody type (SoftMax Pro 5.4).

### Viral Plaque Assay:

Lungs and nasal turbinates will be placed in cold balanced Hanks salt solution supplemented with 1× sucrose phosphate in tissue homogenization tube (MP Biomedicals) and homogenized using an MP FastPrep24 instrument (MP Biomedicals). Clarified supernatant will be serial diluted and placed onto sub-confluent HEp-2 cells in 2 plates. After 90 mm of incubation, supernatants will be removed and cells will be overlaid with MEM supplemented with 0.75% methylcellulose. After 5 days, the medium will be removed and the cells will be fixed with methanol. Plaques will be visualized by immunodetection.

### Cytokine Quantification:

Serum harvested post 6 h of immunization and supernatants from splenocytes stimulated with a RSV F overlapping peptide pool for 48 h will be evaluated for the presence of cytokines in a Luminex-based cytokine profiling assay (Millipore, Billerica, Mass.). Custom kits including INF-IL-5, IL-6, IP-IO, MCP-1, and KC will be used according to the manufacturer protocol and read using a Bio-Rad Luminex 200 reader (Bio-Rad, Hercules, Calif.).

### Cell Mediated Immunity:

Splenocytes will be isolated using standard procedures. From this preparation, the number of mouse splenocytes secreting gamma interferon (IFN-γ) will be determined by enzyme-linked immunospot (ELISPOT) assay (BD Biosciences, San Diego, Calif.) according to the manufacturer's recommendations. For the in vitro stimulation, splenocytes from individual mice (5×10⁷/well) will be incubated with a RSV F-specific CD8 peptide (KYKNAVTEL), or 2 RSV F-specific CD4 peptides (GWYTSVITIELSNJKE and VSVLTSKVLDLKNYI) at a concentration of 1 μg/mL per peptide (IPT, Berlin, Germany). Controls will include splenocytes that are stimulated with Cell Stimulation Cocktail (eBioscience, San Diego, Calif.) or mock stimulated. Following 20 h of incubation in the presence of peptide at 37° C. in a humidified incubator, the ELISPOT assay will be completed and spots will be counted by an ImmunoSpot ELISPOT assay reader (Cellular Technology Ltd., Cleveland, Ohio). For analysis, the spot counts in medium control wells will be subtracted from the specific spot count after peptide stimulation, and the difference reported as the number of spot-forming cells (SFC) per 1×10⁶ splenocytes.

### EXPECTED RESULTS AND CONCLUSIONS

Immunogenicity assessment of the Macrofol-S emulsion, using RSV F as a model antigen, is expected to show that this emulsion trended toward better humoral and cellular immune response compared to the larger sized emulsions provided as conventional commercial emulsion formulations of squalene.

It will be understood that the invention is not restricted to the specific details described above and that numerous modifications and variations can be made without departing from the teachings of the invention as disclosed in the specifications.

What is claimed is:

1. A stable lipophilic drug-containing emulsion comprising:
oil droplets of a mean oil particle diameter of 80-300 nanometers; and
a continuous aqueous phase comprising:
a lipophilic drug in an amount 0.5-5.0% by weight relative to the weight of the total emulsion, wherein said lipophilic drug has a solubility in water of less than 1 mg/mL;
a primary oil physiologically suitable for parenteral administration to a mammal comprising plant-derived biocompatible long chain triglycerides; and
a secondary oil comprising an ethyl ester of a saturated, unbranched carboxylic acid of 4-8 carbon atoms or an unbranched alkyl esters of acetic acid, said alkyl residue having 4-8 carbon atoms, or combination thereof, the combined percentage by weight of the oil components not exceeding about 10 percent.

2. The emulsion of claim 1, wherein said continuous aqueous phase further comprises an ionic surfactant in an amount 0.00001-0.001% by weight relative to the total weight of the emulsion.

3. The emulsion of claim 1, wherein said continuous aqueous phase further comprises a non-ionic synthetic surfactant in an amount 1.0-3.0% by weight relative to the total weight of the emulsion.

4. The emulsion of claim 1, wherein said continuous aqueous phase further comprises a tonicity agent in an amount of 2.0-2.5% by weight relative to the total weight of the emulsion.

5. The emulsion of claim 1, wherein said continuous aqueous phase further comprises water to adjust the concentrations of components to the ranges specified.

6. The emulsion of claim 1 wherein said primary oil is selected from the group consisting of vegetable oils, almond oil, apricot kernel oil, avocado oil, canola oil, hazelnut oil, mustard oil, coconut oil, oat oil, olive oil, peanut oil, rice bran oil, safflower oil, sesame oil, soybean oil, and sunflower oil.

7. The emulsion of claim 1 wherein said secondary oil is butyl acetate, hexyl acetate, octyl acetate, ethyl butyrate, ethyl hexanoate, and ethyl octanoate.

8. The emulsion of claim 1 wherein said secondary oil has a solubility in water of less than 0.75 weight percent, and a solubility of less than 1.0 weight percent water in said secondary oil.

9. The emulsion of claim 1 wherein said ionic surfactant is selected from the group consisting of sodium myristate, sodium palmitate, sodium palmitoleate, sodium stearate, sodium oleate, sodium linoleate, sodium arachidate, and sodium behenate.

10. The emulsion of claim 1 wherein said non-ionic surfactant is a poloxamer having hydroxyl, carboxylate, sulfate, ester, sugar, or amino end-groups.

11. The emulsion of claim 1 wherein said tonicity agent is glycerin, dextrose, or mannitol.

12. The emulsion of claim 1 wherein said lipophilic drug is 99.99 percent partitioned into the oil phase of said emulsion, and is present in the aqueous phase of said total emulsion at a concentration of less than about 15 micrograms per milliliter.

13. The emulsion of claim 1, wherein said emulsion is essentially free of one or more agents selected from the group consisting of EDTA, egg lecithin and benzyl alcohol.

14. The emulsion of claim 1, wherein the pH of said continuous aqueous phase is from pH 4.0 to 8.0.

15. The emulsion of claim 1, wherein said emulsion is stable at room temperature for a period selected from the group consisting of at least 6 months, at least 12 months, at least 18 months and at least 24 months.

16. A method of preparing a stable lipophilic drug-containing emulsion having a lipophilic phase comprising a primary and a secondary oil, and an aqueous phase comprising:
dissolving the lipophilic drug in the primary oil or the secondary oil, or both, and combining into a single oil phase;
combining aqueous stock solutions and the oil phase in predetermined proportions to provide a mixture;
adding water to adjust the concentrations of the components to the desired ranges in said mixture;
and emulsifying said mixture under conditions to obtain an emulsion having a 100 to 300 nanometer mean oil particle diameter.

17. The method of claim 16, wherein said emulsifying further comprises subjecting the oil and water mixture to a first sonication to obtain a coarse emulsion.

18. The method of claim 17, wherein said emulsifying further comprises subjecting the coarse emulsion to a second sonication.

19. The method of claim 16, further comprising dissolving surfactants into the aqueous phase stock solutions with heat and agitation.

20. The method of claim 16, further comprising dissolving a tonicity agent into the aqueous phase stock solution.

21. The method of claim 16, further comprising aliquotting the emulsions into containers and sealing.

22. The method of claim 21, further comprising sterilizing the contents of the containers by conventional means without addition of anti-pathogenic agents.

23. A stable propofol-containing emulsion comprising:
oil droplets of a mean oil particle diameter of 100-300 nanometers and a continuous aqueous phase comprising:
propofol in an amount 0.5-2.0% by weight relative to the weight of the total emulsion;
a primary oil physiologically suitable for parenteral administration to a mammal comprising plant-derived biocompatible long chain triglycerides; and
a secondary oil comprising an ethyl ester of a saturated, unbranched carboxylic acid of 4-8 carbon atoms or an unbranched alkyl ester s of acetic acid, said alkyl residue having 4-8 carbon atoms, or combination thereof, the combined percentage by weight of the oil components not exceeding about 10 percent.

24. The emulsion of claim 23, wherein said continuous aqueous phase further comprises an ionic surfactant in an amount 0.00001-0.001% by weight relative to the total weight of the emulsion.

25. The emulsion of claim 23, wherein said continuous aqueous phase further comprises a non-ionic synthetic surfactant in an amount 1.0-3.0% by weight relative to the total weight of the emulsion.

26. The emulsion of claim 23, wherein said continuous aqueous phase further comprises a tonicity agent in an amount of 2.0-2.5% by weight relative to the total weight of the emulsion.

27. The emulsion of claim 23, wherein said continuous aqueous phase further comprises water to adjust the concentrations of components to the ranges specified.

28. The emulsion of claim 23, wherein said primary oil is selected from the group consisting of vegetable oils, almond oil, apricot kernel oil, avocado oil, canola oil, hazelnut oil, mustard oil, coconut oil, oat oil, olive oil, peanut oil, rice bran oil, safflower oil, sesame oil, soybean oil, and sunflower oil.

29. The emulsion of claim 23, wherein said secondary oil is butyl acetate, hexyl acetate, octyl acetate, ethyl butyrate, ethyl hexanoate, and ethyl octanoate.

30. The emulsion of claim 23, wherein said secondary oil has a solubility in water of less than 0.75 weight percent, and a solubility of less than 1.0 weight percent water in said secondary oil.

31. The emulsion of claim 23, wherein said ionic surfactant is selected from the group consisting of sodium myristate, sodium palmitate, sodium palmitoleate, sodium stearate, sodium oleate, sodium linoleate, sodium arachidate, and sodium behenate.

32. The emulsion of claim 23, wherein said non-ionic surfactant is a poloxamer having hydroxyl, carboxylate, sulfate, ester, sugar, or amino end-groups.

33. The emulsion of claim 23, wherein said tonicity agent is glycerin, dextrose, or mannitol.

34. The emulsion of claim 23, wherein said propofol is 99.99 percent partitioned into the oil phase of said emulsion, and is present in the aqueous phase of said total emulsion at a concentration of less than about 15 micrograms per milliliter.

35. The emulsion of claim 23, wherein said emulsion is essentially free of one or more agents selected from the group consisting of EDTA, egg lecithin and benzyl alcohol.

36. The emulsion of claim 23, wherein the pH of said continuous aqueous phase is from pH 4.0 to 8.0.

37. The emulsion of claim 23, wherein said emulsion is stable at room temperature for a period selected from the group consisting of at least 6 months, at least 12 months, at least 18 months and at least 24 months.

38. A method of preparing a stable propofol-containing emulsion having a lipophilic phase comprising a primary and a secondary oil, and an aqueous phase comprising:
dissolving the propofol in the primary oil or the secondary oil, or both, and combining into a single oil phase;
combining aqueous stock solutions and the oil phase in predetermined proportions to provide a mixture;
adding water to adjust the concentrations of the components to the desired ranges in said mixture;
and emulsifying said mixture under conditions to obtain an emulsion having a 100 to 300 nanometer mean oil particle diameter.

39. The method of claim 38, wherein said emulsifying further comprises subjecting the oil and water mixture to a first sonication to obtain a coarse emulsion.

40. The method of claim 39, wherein said emulsifying further comprises subjecting the coarse emulsion to a second sonication.

41. The method of claim 38, further comprising dissolving surfactants into the aqueous phase stock solutions with heat and agitation.

42. The method of claim 38, further comprising dissolving a tonicity agent into the aqueous phase stock solution.

43. The method of claim 38, further comprising aliquotting the emulsions into containers and sealing.

44. The method of claim 43, further comprising sterilizing the contents of the containers by conventional means without addition of anti-pathogenic agents.
